(19) United States
(12) Patent Application Publication
(10) Pub. No.: US 2026/0232774 A1
(43) Pub. Date: Aug. 13, 2026

(54) COMPOSITIONS AND METHODS FOR TREATING INTESTINAL MICROBIOME DYSBIOSIS

(71) Applicant: The Regents of the University of Michigan, Ann Arbor, MI (US)

(72) Inventor: Pavan Reddy, Ann Arbor, MI (US)

(21) Appl. No.: 19/152,500

(22) PCT Filed: Jan. 30, 2024
(86) PCT No.: PCT/US2024/013526

§ 371 Date: Jul. 31, 2025

Related U.S. Application Data
(60) Provisional application No. 63/442,178, filed on Jan. 31, 2023.

(51) Int. Cl. A61K 38/46; A61K 31/16; A61K 31/198; A61K 31/4196; A61K 31/426; A61K 31/473; A61K 33/242; A61K 38/40; A61K 45/06; A61P 1/00; C07K 16/28

(52) U.S. Cl. CPC A61K 38/465; A61K 31/16; A61K 31/198; A61K 31/4196; A61K 31/426; A61K 31/473; A61K 33/242; A61K 38/40; A61K 45/06; A61P 1/00; C07K 16/2875; C12Y 301/03002; C12Y 301/03016

(57) ABSTRACT

This invention relates generally to compositions and methods for treating, preventing, and/or ameliorating symptoms characterized with intestinal microbiome dysbiosis through restoring intestinal physiological hypoxia in subjects suffering from intestinal microbiome dysbiosis. In particular, the present invention provides methods for decreasing dysbiosis, restoring the microbiome, and/or increasing recovery of a microbiome (e.g., following a dysbiosis inducing event), by administering pharmaceutical compositions to a subject capable of restoring intestinal physiological hypoxia.

### RELATED APPLICATIONS

This application claims priority to U.S. Provisional Patent Application No. 63/442,178, filed Jan. 31, 2023, the entire contents of which are incorporated herein by reference for all purposes.

### STATEMENT REGARDING FEDERALLY SPONSORED RESEARCH OR DEVELOPMENT

This invention was made with government support under HL152605, CA217156 and HL149633 awarded by the National Institutes of Health. The government has certain rights in the invention.

### SEQUENCE LISTING

The text of the computer readable sequence listing filed herewith, titled “UM_41632_601_SequenceListing.xml”, created Jan. 30, 2024, having a file size of 6,354 bytes, is hereby incorporated by reference in its entirety.

### FIELD OF THE INVENTION

This invention relates generally to compositions and methods for treating, preventing, and/or ameliorating symptoms characterized with intestinal microbiome dysbiosis through restoring intestinal physiological hypoxia in subjects suffering from intestinal microbiome dysbiosis. In particular, the present invention provides methods for decreasing dysbiosis, restoring the microbiome, and/or increasing recovery of a microbiome (e.g., following a dysbiosis inducing event), by administering pharmaceutical compositions to a subject capable of restoring intestinal physiological hypoxia.

### BACKGROUND OF THE INVENTION

The composition of host intestinal microbiome directly contributes to human health and diseases. Correspondingly, a healthy gut is inhabited by a diverse community of mostly obligate anaerobic bacteria (eubiosis) that is influenced by the host genetics, diet and immunity^{1 2}. The breakdown of this balance, called dysbiosis and often characterized by a shift in the microbial community structure from obligate to facultative anaerobes and aerotolerant microbes, is associated with several diseases, including immune mediated intestinal diseases such as graft-versus-host disease (GVHD)^{3 4 5 6}. Whilst a strong correlation exists between disease severity and dysbiosis, the mechanisms that lead to dysbiosis are unknown. Furthermore, whether dysbiosis is a cause, an amplifier, a regulator or a mere consequence of the disease process remains poorly understood.

Gastrointestinal (GI) GVHD is a potentially fatal complication of allogeneic hematopoietic stem cell transplantation (HSCT)⁷. Reduction in intestinal microbial diversity with loss of obligate anaerobes relative to other bacteria is associated with increased GVHD mortality^{3 8 9}. However, seminal experimental studies performed before the advent of modern germ-free facilities and sequencing technologies demonstrated that absence of microbiome mitigated GVHD severity¹⁰. Thus, while microbial dysbiosis prognosticates outcomes after allogeneic (allo) HSCT, the role of microbiome itself in GVHD remains unclear. Specifically, (a) whether dysbiosis is caused by or is a consequence of severe GVHD, (b) the mechanisms that underpin the development of dysbiosis, and (c) whether dysbiosis before or after HSCT directly amplifies or negatively regulates GVHD severity remain unknown.

The mammalian GI tract is hypoxic with an oxygen (O₂) gradient that traverses the surface of the colonic mucosa to the center of gut lumen. This physiologic hypoxic gradient shapes the intestinal microbial community structure, promoting colonization with predominantly obligate anaerobes at homeostasis^{11 12}. The host intestinal epithelial cells (IECs) are thus uniquely adapted to the hypoxic environment, i.e., “physiologic hypoxia”. The IECs depend on microbial metabolites, specifically short-chain fatty acids (SCFAs), as their primary source of energy for oxidative phosphorylation (OXPHOS) despite low oxygen^{13 14}. Thus, microbial-derived SCFAs regulate the barrier function at homoeostasis¹⁵ and in GI GVHD^{16 17}. Moreover, epithelial metabolism and the cellular O₂ sensor, hypoxia-inducible factor1α (HIF), are key determinants of intestinal function under conditions of “physiologic hypoxia”^{1 18}. In the context of enteric infections, intestinal ambient oxygen level is a critical ecological driver of dysbiosis^{1 2 19 20}. In the context of non-infectious immune mediated GI damage, pathogenic T cells target OXPHOS in the IEC leading to deficient O₂ utilization²¹. However, whether the change in the luminal oxygen due to poor consumption of O₂ from the metabolic defect has an impact on dysbiosis remains unknown.

The present invention addresses these unresolved issues.

### SUMMARY

Experiments conducted during the course of developing embodiments for the present invention demonstrated that dysbiosis was a consequence of the loss of ‘physiologic hypoxia’ from poor utilization of oxygen caused by alloreactive T cell-mediated metabolic defect in the IECs²¹. Such experiments further demonstrated that the microbiome by itself is a critical negative regulator of GI GVHD because germ-free animals, in contrast to the current paradigm, demonstrated greater GVHD while promotion of eubiosis after transplant mitigated the severity of GVHD. By contrast, presence of dysbiosis before HSCT did not impact GVHD severity. It was further demonstrated that rescue of physiological hypoxia by iron chelation mitigated the severity of GI GVHD. Thus, the results of such experiments described herein provide a mechanism for induction of dysbiosis and demonstrate that promotion of eubiosis by regulating ambient oxygen reduces the severity of intestinal damage after allo-HCT.

Accordingly, the present invention relates generally to compositions and methods for treating, preventing, and/or ameliorating symptoms characterized with intestinal microbiome dysbiosis through restoring intestinal physiological hypoxia in subjects suffering from intestinal microbiome dysbiosis. In particular, the present invention provides methods for decreasing dysbiosis, restoring the microbiome, and/or increasing recovery of a microbiome (e.g., following a dysbiosis inducing event), by administering pharmaceutical compositions to a subject capable of restoring intestinal physiological hypoxia.

In certain embodiments, the present invention provides methods for decreasing conditions associated with impaired intestinal physiological hypoxia in a subject comprising administering to the subject a therapeutically effective amount of a pharmaceutical composition comprising one or more agents capable of restoring intestinal physiological hypoxia.

In certain embodiments, the present invention provides methods for decreasing intestinal dysbiosis in a subject comprising administering to the subject a therapeutically effective amount of a pharmaceutical composition comprising one or more agents capable of restoring intestinal physiological hypoxia.

In certain embodiments, the present invention provides methods for restoring intestinal eubiosis (e.g., restoring a healthy microbiome) in a subject comprising administering to the subject a therapeutically effective amount of a pharmaceutical composition comprising one or more agents capable of restoring intestinal physiological hypoxia.

In certain embodiments, the present invention provides methods for increasing the recovery of a healthy microbiome in a subject after a dysbiosis inducing event comprising administering to the subject a therapeutically effective amount of a pharmaceutical composition comprising one or more agents capable of restoring intestinal physiological hypoxia.

In certain embodiments, the present invention provides methods for protecting the microbiome in a subject (e.g., protecting the subject's microbiome following exposure to a dysbiosis inducing event) comprising administering to the subject a therapeutically effective amount of a pharmaceutical composition comprising one or more agents capable of restoring intestinal physiological hypoxia.

In certain embodiments, the present invention provides methods for treating GvHD in a subject comprising administering to the subject a therapeutically effective amount of a pharmaceutical composition comprising one or more agents capable of restoring intestinal physiological hypoxia.

In certain embodiments, the present invention provides methods for treating inflammatory bowel disorder in a subject comprising administering to the subject a therapeutically effective amount of a pharmaceutical composition comprising one or more agents capable of restoring intestinal physiological hypoxia.

Such embodiments are not limited to a particular type or kind of subject. In some embodiments, the subject is a human subject. In some embodiments, the subject is a mammalian subject.

Such embodiments are not limited to a specific type or kind of condition associated with impaired intestinal physiological hypoxia. In some embodiments, the condition associated with impaired intestinal physiological hypoxia is intestinal dysbiosis. In some embodiments, the condition associated with impaired intestinal physiological hypoxia is an immune mediated intestinal disease. In some embodiments, the immune mediated intestinal disease is selected from, for example, idiopathic inflammatory bowel disease (IBD), graft versus host disease (GVHD), and enteropathy in primary immunodeficiency. In some embodiments, the condition associated with impaired intestinal physiological hypoxia is related to allogeneic hematopoietic stem cell transplantation (HSCT). In some embodiments, the condition associated with impaired intestinal physiological hypoxia is associated with decreased obligate anaerobes in the subject's microbiome. In some embodiments, the condition associated with impaired intestinal physiological hypoxia is associated with T-cell mediated metabolic defect in intestinal epithelial cells. In embodiments, the condition associated with impaired intestinal physiological hypoxia is related to a dysbiosis inducing event. In some embodiments, the dysbiosis inducing event is treatment with one or more antibiotics (e.g., vancomycin) in connection with surgery. In some embodiments, the dysbiosis inducing event is an infectious disease. In some embodiments, the dysbiosis inducing event is infection by Clostridium difficile. In some embodiments, the dysbiosis inducing event is a primary infection by Clostridium difficile. In some embodiments, the dysbiosis inducing event is a secondary or recurring infection by Clostridium difficile. In some embodiments, the dysbiosis inducing event is traveler's diarrhea.

Such embodiments are not limited to a specific type or kind of agent capable of restoring intestinal physiological hypoxia. In some embodiments, the agent capable of restoring intestinal physiological hypoxia is an iron binding protein. In some embodiments, the iron binding protein is selected from the group consisting of lactoferrin, transferrin, ferritin, Ferric uptake repressor (FUR) protein, calcineurin, acid phosphatase and ferredoxin. In some embodiments, the iron binding protein is an iron chelator. In some embodiments, the iron chelator is selected from the group consisting of desferrioxamine, Deferasirox, FBS0701, phenanthroline, ethylene diamine tetra-acetic acid (EDTA), diethylene triamine-pentaacetic acid (DTPA) and N,N′-bis[2-hydroxybenzoyl]ethylene diamine-N,N′-diacetic acid (HBED). In some embodiments, the agent capable of restoring intestinal physiological hypoxia is any pharmaceutic agent capable of restoring intestinal eubiosis in the subject.

Such embodiments are not limited to a particular manner of administering to the subject an agent capable of restoring intestinal physiological hypoxia. In some embodiments, the agent capable of restoring intestinal physiological hypoxia is administered orally. In some embodiments, the agent capable of restoring intestinal physiological hypoxia is administered by oral gavage.

In some embodiments, such methods further comprises administering to the subject one or more of the following additional therapeutic agents: disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors. In some embodiments, the therapeutic agents include, but are not limited to, infliximab, adalimumab, etanercept, parenteral gold or oral gold.

In certain embodiments, the present invention provides a composition comprising one or more agents capable of restoring intestinal physiological hypoxia.

In certain embodiments, the present invention provides a pharmaceutical composition comprising one or more agents capable of restoring intestinal physiological hypoxia.

The present invention also provides kits comprising one or more agents capable of restoring intestinal physiological hypoxia and other therapeutic agents.

### BRIEF DESCRIPTION OF THE DRAWINGS

FIG. 1A-N. Allogeneic dysbiosis is not pathogenic to naïve animals. (A to F), naïve C57BL/6 mice (B6) 7-8 weeks old were co-housed with allogeneic recipient mice (Allo B6) two weeks after post-transplant. Mice were co-housed in a ratio of 1:1 naïve mice to BMT mice respectively (A). Data are from 3 independent experiments (N=9). Stool from Allo B6 before co-house or B6 were analyzed by 16S rRNA gene sequencing. (B to D) Microbiome composition (left) with the percent abundance of obligate anaerobes and other bacteria (right) (B), PCoA (C), and inverse Simpson alpha diversity index of microbiome (D) in stool were shown. (E and F) The body weigh change (H) and the clinical GVHD score (F) of B6 co-housed with Allo B6, not co-housed B6, and not co-housed Allo B6 are shown (co-housed with Allo B6 and not co-housed B6: N=9, not co-housed Allo B6; N=6). (G to L) B6 mice 6-8 weeks old were treated with 2 weeks of 4 antibiotics cocktail (ampicillin 1 gr/L, kanamycin 1 gr/L, metronidazole 1 gr/L and vancomycin 0.5 gr/L plus 3% stevia) in filtered double distillated drinking water. Antibiotics treated mice (B6Ab) were co-housed in ratio of 1:1 antibiotic treated mice to BMT mice respectively (G). Data are from 3 independent experiments. (H) Stool from B6Ab, B6 and Germ free (GF) mice were analyzed by qPCR. Relative quantification of bacteria in stool from B6, antibiotics treated B6 at day7, and GF mice were analyzed (B6: N=10, Antibiotics d7: N=10, GF: N=8). (I) Inverse Simpson alpha diversity index of microbiome of B6 and B6Ab are shown (B6N: N=4, B6Ab, N=3). (J) Inverse Simpson alpha diversity index of microbiome of B6Ab, B6Ab co-housed with Syn or Allo B6 at day14 after co-house were shown (B6Ab N=4, B6Ab co-housed with Syn or Allo B6 N=2). (K) The body weight changes of co-housed B6Ab mice are shown (Co-housed with Syn: N=14, Co-housed with Allo: N=15). (L) Ileal and colonic histopathological GVHD score of co-housed B6Ab mice are shown (N=3). (M and N) B6 mice were treated for 2 weeks with 4 antibiotics cocktail (ampicillin 1 mg/ml, neomycin 1 mg/ml, metronidazole 1 mg/ml and vancomycin 0.5 mg/ml) in filtered double distillated drinking water, followed by 10 doses of intestinal content gavage from BMT recipient mice 2 weeks after BMT. Each gavage day one BMT mouse whole gut content was collected. Stool solution was gavaged to each recipient mouse (M). Data are from 3 independent experiments (B6Ab with Allo stool: N=15, B6Ab with PBS: N=8). (N) The body weight change of gavaged B6Ab mice are shown. The horizontal line in box (B, D, I, J) represents the median with the box bounding the interquartile range. The ends of the whisker lines represent the minimum and maximum values. Two-tailed paired Wilcoxon test (B) and Two-tailed unpaired t-test (D, I, L), and one-way ANOVA analysis with Tukey post hoc test (H, J) were used to determine significance (mean±s.e.m.). *P<0.05, **P<0.01, ***P<0.001, ****P<0.0001. See also FIGS. 2 and 3.

FIG. 2A-G: The microbiome composition of stool from B6 and dysbiotic B6 mice. The details of the experimental design were shown in FIG. 1A. (A and B) Stool from B6 and Allo B6 2 weeks after BMT were analyzed by 16S rRNA gene sequencing. A cladogram (A) and LDA scores (B) are shown for taxa differentially abundant by LEfSe analysis. (C to G) Stool from Allo B6 before co-house and B6 co-housed with Allo B6 (2 weeks and 6 weeks after co-house) analyzed by 16S rRNA gene sequencing. PCoA (C), inverse Simpson alpha diversity index of microbiome composition (D), microbiome composition (E), and taxa differentially abundant by LEfSe analysis (F & G) are shown (C, Allo B6:N=7, B6 co-housed with Allo B6, 6 week:N=6, B6: N=5, B6Ab; N=5) (B, Allo B6:N=5, B6 co-housed with Allo B6, 6 week:N=4, B6: N=4, B6Ab; N=3) The horizontal line in box (D) represents the median with the box bounding the interquartile range. The ends of the whisker lines represent the minimum and maximum values. One-way ANOVA analysis with Tukey post hoc test (D) was used to determine significance.

FIG. 3A-F: The microbiome composition of B6Ab and B6Ab gavaged Allo B6 stool. B6 mice were treated for 2 weeks with 4 antibiotics cocktail (B6Ab, ampicillin 1 mg/ml, neomycin 1 mg/ml, metronidazole 1 mg/ml and vancomycin 0.5 mg/ml). Stool from B6Ab and B6 were analyzed by 16S rRNA gene sequencing. (A and B) PCoA (A), and microbiome composition (B) in stool from B6 and B6Ab were shown (B6N: N=7, B6Ab, N=5). (C and D) B6 received BMT from B6 (Syn) or BALB/c (Allo) donor. Stool from B6, Syn B6, and Allo B6 day7 after BMT were analyzed by 16S rRNA gene sequencing. PCoA (C) and inverse Simpson alpha diversity index of microbiome composition (D) in stool were shown. (B6: N=9, Syn B6: N=5, Allo B6: N=4) (E and F) The details of the experimental design were shown in FIG. 1M. The inverse Simpson alpha diversity index of microbiome composition (E), microbiome composition (F) of stool from Allo B6 2 weeks after BMT and B6Ab gavaged allogeneic intestinal content (2 week) are shown (Allo B6: N=9, B6Ab with Allo stool, 2 week: N=3). The horizontal line in box (D, E) represents the median with the box bounding the interquartile range. The ends of the whisker lines represent the minimum and maximum values. One-way ANOVA analysis with Tukey post hoc test (D) and two-tailed unpaired t-test (E) were used to determine significance. *P<0.05, **P<0.01.

FIG. 4A-E: Systematic phenotyping of T cells and dendritic cells in lymphoid tissue and intestine. B6 mice were treated for 2 weeks with 4 antibiotics cocktail (ampicillin 1 mg/ml, neomycin 1 mg/ml, metronidazole 1 mg/ml and vancomycin 0.5 mg/ml), followed by 10 doses of intestinal content gavage from BMT recipient mice 2 weeks after BMT. Each gavage day one mouse whole intestinal content was collected and homogenized in sterile PBS. (A and B) Immune profiles of systemic lymph node, spleen, and mesenteric lymph node from mice at day 14 (A) and day 42 (B) after stool gavage are shown. (C) The immune profiles of T cells from colon and ileum of mice at day 14 after stool gavage. (D and E) The pathological GVHD score of ileum and colon from stool donor mice day14 after BMT and mice at day 14 (D) and day 42(E) after stool gavage (Allo B6: N=5, Syn B6: N=4, B6Ab with Syn stool, Day14: N=3, Day42: N=4, B6Ab with Allo stool, Day14: N=3, Day42: N=5, B6Ab with PBS, Day14: N=3, Day42: N=4, B6Ab no Gavage, Day14: N=3, Day42: N=5). One-way ANOVA analysis with Tukey post hoc test (D, E) was used to determine significance (mean±s.e.m.). ****P<0.0001.

FIG. 5A-F: Pre-transplant dysbiosis or eubiosis does not have significant impact on GVHD. (A) B6 mice were treated for 2 weeks with 4 antibiotics cocktail (FIG. 1G) or PBS, followed by gavaged Allo B6 intestinal content or PBS. After antibiotics treatment and intestinal content gavage, mice received BMT. (B and C) Stool from BMT recipients and B6 mice were analyzed by 16S rRNA gene sequencing. PCoA (B) and inverse Simpson alpha diversity index of microbiome (C) are shown. (B6 N=5, Allo N=3, Allo with Ab N=4, Allo with Ab+stool N=4). Survival rate (D), clinical GVHD score (E), and pathological GVHD score of colon (F) are shown. (D) Allo: N=3, Allo with Ab: N=4, Allo with Ab+stool: N=4, B6: N=5. (E) Syn with Ab+stool: N=6, Allo: N=5, Allo with Ab: N=5, Allo with stool: N=5, Allo with Ab+stool: N=6. (F) Syn with Ab+stool: N=3, Allo: N=3, Allo with Ab: N=3, Allo with stool: N=3, Allo with Ab+stool: N=2. Two independent experiments were performed. One-way ANOVA analysis with Tukey post hoc test (C) and one-way ANOVA analysis with Dunn's post hoc test (F) was used to determine significance (mean±s.e.m.). **P<0.01, ***P<0.001. See also FIG. 4.

FIG. 6A-I: Post-transplant eubiosis ameliorates the severity of GVHD. Allo B6 were co-housed with B6 at ratio of Allo B6: B6=1:1 from day 14 after BMT (A). Data are from three independent experiments. (B-F) Stool from Allo B6 co-housed with B6 for 2 weeks, B6 co-housed with Allo B6 for 2 weeks, Allo B6 not co-housed, and B6 were analyzed by 16S rRNA gene sequencing. (B) Inverse Simpson alpha diversity index were shown. (Allo B6 not co-housed: N=6, Allo B6 co-housed with B6: N=4, B6 co-housed with Allo B6: N=5, B6: N=4). (C) PCoA were shown (Allo B6 not co-housed: N=9, Allo B6 co-housed with B6: N=6, B6 co-housed with Allo B6: N=5, B6: N=7). Microbiome composition (D) and taxa differentially abundant by LEfSe analysis (E & F) are shown. Survival rate (G), body weight change (H), and clinical GVHD score (I) of BMT recipients were shown. (Allo B6 were co-housed with B6: N=18, Allo B6 not co-housed: N=6). The horizontal line in box (B) represents the median with the box bounding the interquartile range. The ends of the whisker lines represent the minimum and maximum values. One-way ANOVA analysis with Tukey post hoc test (B), log-rank test (G), and two-tailed Mann-Whitney test (I) was used to determine (mean±s.e.m.). *P<0.05.

FIG. 7A-I: GF mice transplanted with healthy microbiome showed reduced GVHD (A to C) Germ free B6 (GF) mice were gavaged intestinal content from Syn and Allo BMT recipient mice. (A) Body weight change is shown (GF: N=2, GF with Syn B6 stool: N=4, GF with Allo B6 stool: N=5). Stool from GF with Syn or Allo B6 stool 4 weeks after stool gavage analyzed by 16S rRNA gene sequencing. PCoA (B) and microbiome composition (C) are shown (GF with Syn B6 stool: N=7, GF with Allo B6 stool: N=10). (D to F) SPF B6 and GF B6 mice received BMT from B6 or BALB/c donor. Survival rate (D), body weight change (E), and clinical GVHD score (F) are shown (GF Syngeneic: N=2, GF Allogeneic: N=18, SPF Syngeneic: N=2, SPF Allogeneic: N=17). (G to J) GF B6 mice received BMT from B6 or BALB/c donor. Then GF mice were gavaged intestinal content from SPF Syn and SPF Allo BMT recipient mice (G). Survival rate (H) and clinical GVHD score 6 weeks after BMT (I) are shown (Allo GF with Syn B6 stool N=6, Allo GF with Allo B6 stool N=4). (J) Ileum and colonic histopathological score at day7 after BMT are shown (n=4). Two-tailed unpaired t-test (A, I, J), log-rank test (D, H), and two-tailed Mann-Whitney test (F) was used to determine (mean±s.e.m.). *P<0.05, ***P<0.001, ****P<0.0001. See also FIG. 8.

FIG. 8A-D: The microbiome composition of allogeneic GF mice with Syn or Allo B6 stool. (A) The details of the experimental design were shown in FIG. 7A to C. Stool from colon and ileum from GF with Syn or Allo B6 stool at day7 (left) and day14 (right) after stool gavage analyzed by 16S rRNA gene sequencing. PCoA were shown(n=4). (B to D) The details of the experimental design were shown in FIG. 7G. Stool from Allo GF with Syn or Allo B6 stool 2 weeks after intestinal content gavage analyzed by 16S rRNA gene sequencing. (B to D) PCoA (B), inverse Simpson alpha diversity index of microbiome composition (C), and microbiome composition (D) are shown (Allo GF with Syn B6 stool: N=4, Allo GF with Allo B6 stool: (B) N=3, (C) N=5). Two-tailed unpaired t-test (C) (mean±s.e.m.) was used to determine significance. *P<0.05.

FIG. 9A-H: The defect in 02 utilization in IECs leading to a loss of intestinal luminal and cellular physiological hypoxia. (A) Representative bio-energetic profiles of isolated colonic IECs from syngeneic and allogeneic mice (BALB/c→B6) under basal conditions and following treatment with mitochondrial inhibitors (oligomycin, FCCP, rotenone/antimycin A) by Seahorse analyzer. Oxygen consumption rate (OCR) of day21 after BMT is shown (N=4). Four independent experiments were performed. (B) Quantification of 02 levels in intestine from recipients 7 days and 21 days after BMT (BALB/c→B6). Naive: N=5, Syngeneic: N=4 (Day7 outer and Day21 outer), N=5(Day7 inner), Allogeneic: N=4 (Day7 outer and Day21 outer), N=5 (Day7 inner). (C) B6 received BMT from B6 or BALB/c donor. Representative images of Hypoxyprobe staining and relative fluorescent intensity in colon from recipients 21 days after BMT (scale bar=50 μm). (D) Unirradiated B6D2F1 mice received 10×10⁷ splenocytes from syngeneic B6D2F1 or allogeneic B6 donors. Representative images of Hypoxyprobe staining and relative fluorescent intensity in colon from recipients at 21 days after BMT (scale bar=50 μm) (N=4). (E) B6 mice received chemotherapy and received 1×10⁷ T cells along with 1×10⁷ TCD-BM cells from either syngeneic B6 or allogeneic BALB/c donors. Representative images of Hypoxyprobe staining and relative fluorescent intensity in colon from recipients 21 days after BMT (scale bar=50 μm) (N=4). (F) B6 mice received 10 Gy total body irradiation without T-cell and BM cells. Representative images of Hypoxyprobe staining relative fluorescent intensity in colon from recipients 7 days after BMT (scale bar=50 μm) (N=4). (G and H) CD4++CD25−CD4+4−CD4+5RBhi (CD4+5RB high) T cells or CD4++CD25−CD4+4−CD4+5RBlow (CD4+5RB low) T cells from B6 mice were transferred to Rag1−/− SPF (G) or Rag1−/− GF mice (H). Hypoxyprobe staining and relative fluorescent intensity in colon from recipients 8 weeks after T cell transferring (scale bar=50 μm) (SPF: N=4, GF CD4+5RB low: N=4, GF CD4+5RB high N=5). (C to H) At least, four independent experiments were performed. One-way ANOVA analysis with Tukey post hoc test (B) and two-tailed unpaired t-test (C to H) was used to determine significance (mean±s.e.m.). *P<0.05, **P<0.01. See also FIG. 10.

FIG. 10A-H: The defect of 02 utilization in intestine after allo-HSCT. (A) Maximum OCR/extracellular acidification rate (ECAR) ratio of isolated colonic IECs from syngeneic and allogeneic mice (BALB/c→B6) at day21 after BMT are shown (N=4). (B and C) The details of experimental design is in FIG. 9D. Clinical GVHD score (B) and survival rate (C) were shown (Syngeneic: N=6, Allogeneic: N=19). (D to F) Taconic B6 (D), Taconic BALB/c (E), and JAX BDF1(F) mice received BMT as described in Methods. Representative image of flowcytometry with Hypoxyprobe-APC and mean fluorescent intensity in syngeneic and allogeneic mice at day7 after BMT were shown. (G and H) The details of the experimental design were shown in FIGS. 9G and H. Body weigh change of SPF mice (G) and GF mice(H) were shown (SPF CD45RB low: N=4, SPF CD45RB high: N=6, GF CD45RB low: N=4, GF CD45RB high: N=6). Two-tailed unpaired t-test (A, D to H) and two-tailed Mann-Whitney test (B) (mean±s.e.m.) were used to determine significance. *P<0.05, **P<0.01, ***P<0.001, ****P<0.0001.

FIG. 11A-C: Loss of hypoxia in colon and dysbiosis are induced by GVHD with independent of the different institution and microbiome. Lethally irradiated (1000 cGy) 129 mice received bone marrow (5×10⁶ cells) and purified CD5⁺T cells (2×10⁶ cells) from C57BL/6J donors in different institution (Memorial Sloan Kettering Cancer Center). (A) Tissue hypoxia was determined with Pimonidazole (left). Quantification of intestinal O₂ levels in intestine from recipients (right). Samples were collected 4, 8, 14, and 21 days after BMT (Naïve: N=3, BM only: N=4, BM+T: day4, 8, 14 N=4, day21 N=3). (B) Stool microbial composition was determined by 16S rRNA gene sequencing. (C) The classification of obligate/facultative (0/F) anaerobe ratio was determined. Two-tailed unpaired t-test (A) was used to determine significance (mean±s.e.m.). **P<0.01.

FIG. 12A-L: 02 modulation after allo-HSCT improves intestinal physiologic hypoxia and GVHD. (A to D) B6 received BMT from syngeneic B6 or allogeneic BALB/c donor. (A) Hif1α mRNA expression of isolated colonic IECs from BMT recipients 21 days after BMT (N=4). (B) Representative image and the relative intensity of HIF1α immunohistochemical staining with intestinal tissue from BMT recipients 21 days after BMT are shown (Scale bar=50 μm, N=4). (C) Egln3 (Phd3) mRNA expression of isolated colonic IECs from BMT recipients on day21 after BMT (N=3). (D) Representative image of immunoblot and the relative protein density for PHD3 and β-Actin with colonic IECs from BMT recipients 21 days after BMT are shown (N=4). (E and F) Hif1αfl/fl mice and Hif1αfl/fl Vil1cre mice received BMT from BALB/c donor. Survival rate (E) and clinical GVHD (F) score are shown (Hif1α^{fl/fl} allogeneic: N=6, Hif1α^{fl/fl} Vil1cre: N=4). (G to L) B6 received BMT from BALB/c donor mice. BMT recipients were orally treated with deferasirox (20 mg/kg) and vehicle every day. (G) Hypoxyprobe staining and relative fluorescent intensity in colon from recipients 21 days after BMT (scale bar=50 μm). Four independent experiments were performed. Stool from BMT recipients were analyzed by 16S rRNA gene sequencing. PCoA (H), inverse Simpson alpha diversity index of microbiome (I), and microbiome composition (J) in stool from recipients 21 days after BMT were shown (Allo B6 vehicle: N=7, Allo B6 deferasirox: N=9). Survival rate (K) and clinical GVHD score (L) of BMT recipients (Allo B6 vehicle: N=24, Allo B6 deferasirox: N=10). Representative plots and a graph summarizing the results of at least two independent experiments are shown. Two-tailed unpaired t-test (A, C, G, T), two-tailed Mann-Whitney test (B, D, F, L), or log-rank test (E, K) (mean±s.e.m.) were used to determine significance. *P<0.05, **P<0.01. See also FIG. 13.

FIG. 13A-F: Iron chelator treatment does not alter T cells function. (A) The details of the experimental design were shown in FIG. 12G to L. Quantification of iron in stool from colon and ileum in Allo B6 and Allo B6 deferasirox at 7 day and 21 day after BMT were shown (Day7, N=2, Day21, Vehicle: N=4, Deferasirox: N=2). (B) The percent of IFNγ+CD4, IFNγ+CD8, and Treg cells in spleen from recipients at day7 after BMT were shown (N=4). (C) The cytokine levels of IFNγ, TNFα, and IL17A in serum from recipients at day7 after BMT were shown (Vehicle: N=3, Deferasirox: N=4). (D to F) GF mice received BMT from BALB/c donor mice. BMT recipients were orally treated with deferasirox (20 mg/kg) and vehicle every day. (D) Hypoxyprobe staining and relative fluorescent intensity in colon from recipients 21 days after BMT (scale bar=50 μm). Three independent experiments were performed. Survival rate (E) and clinical GVHD score (F) of BMT recipients (Syn GF vehicle: N=2, Syn GF deferasirox: N=2, Allo GF vehicle: N=8, Allo GF deferasirox: N=10). The horizontal line in box (C) represents the median with the box bounding the interquartile range. The ends of the whisker lines represent the minimum and maximum values. Two-tailed unpaired t-test (A, B, C), log-rank test (E), or two-tailed Mann-Whitney test (F) was used to determine significance (mean±s.e.m.). *P<0.05, **P<0.01, ***P<0.001.

### DEFINITIONS

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

The present invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The transitional term “comprising,” which is synonymous with “including,” “containing,” or “characterized by,” is inclusive or open-ended and does not exclude additional, unrecited elements or method steps. By contrast, the transitional phrase “consisting of excludes any element, step, or ingredient not specified in the claim. The transitional phrase “consisting essentially of limits the scope of a claim to the specified materials or steps “and those that do not materially affect the basic and novel characteristic(s)” of the claimed invention.

As used herein, “dysbiosis” refers to an imbalance in the microbiome within a subject or on the surface of the subject. In dysbiosis, the normal microbiome of the subject is perturbed or damaged, which may lead to a variety of diseases and/or disorders. Dysbiosis may result, for example, from a loss of beneficial species, loss of microbial diversity, increase in pathogenic organism(s), and/or change in metabolic capacity. As used herein, “decreasing dysbiosis” refers to restoring the microbiota community composition and homeostasis.

A “subject” can be a vertebrate, a mammal, or a human. Mammals include, but are not limited to, farm animals, sport animals, pets, primates, mice and rats. In one aspect, a subject is a human.

Unless otherwise defined, all technical terms used herein have the same meaning as commonly understood by one of ordinary skill in the art to which this disclosure belongs.

### DETAILED DESCRIPTION

The severity of T-cell mediated gastro-intestinal (GI) diseases such as graft-versus-host host disease (GVHD) and inflammatory bowel diseases correlate with a decrease in the diversity of the host gut microbiome composition characterized by loss of obligate anaerobic commensals. The mechanisms underpinning these changes in the microbial structure remain unknown. Experiments described herein demonstrate that in multiple specific pathogen free (SPF), gnotobiotic and germ-free murine models of GI GVHD that the initiation of the intestinal damage by the pathogenic T cells altered ambient oxygen levels in the GI tract and caused dysbiosis. The change in oxygen levels contributed to the severity of intestinal pathology in a host intestinal HIF-1α- and a microbiome-dependent manner. Regulation of intestinal ambient oxygen levels with oral iron chelation mitigated dysbiosis and reduced the severity of the GI GVHD. Thus, targeting ambient intestinal oxygen levels may represent a novel, non-immunosuppressive strategy to mitigate T-cell driven intestinal diseases.

Accordingly, the present invention relates generally to compositions and methods for treating, preventing, and/or ameliorating symptoms characterized with intestinal microbiome dysbiosis through restoring intestinal physiological hypoxia in subjects suffering from intestinal microbiome dysbiosis. In particular, the present invention provides methods for decreasing dysbiosis, restoring the microbiome, and/or increasing recovery of a microbiome (e.g., following a dysbiosis inducing event), by administering pharmaceutical compositions to a subject capable of restoring intestinal physiological hypoxia).

In certain embodiments, the present invention provides methods for decreasing conditions associated with impaired intestinal physiological hypoxia in a subject comprising administering to the subject a therapeutically effective amount of a pharmaceutical composition comprising one or more agents capable of restoring intestinal physiological hypoxia.

In certain embodiments, the present invention provides methods for decreasing intestinal dysbiosis in a subject comprising administering to the subject a therapeutically effective amount of a pharmaceutical composition comprising one or more agents capable of restoring intestinal physiological hypoxia.

In certain embodiments, the present invention provides methods for restoring intestinal eubiosis (e.g., restoring a healthy microbiome) in a subject comprising administering to the subject a therapeutically effective amount of a pharmaceutical composition comprising one or more agents capable of restoring intestinal physiological hypoxia.

In certain embodiments, the present invention provides methods for increasing the recovery of a healthy microbiome in a subject after a dysbiosis inducing event comprising administering to the subject a therapeutically effective amount of a pharmaceutical composition comprising one or more agents capable of restoring intestinal physiological hypoxia.

In certain embodiments, the present invention provides methods for protecting the microbiome in a subject (e.g., protecting the subject's microbiome following exposure to a dysbiosis inducing event) comprising administering to the subject a therapeutically effective amount of a pharmaceutical composition comprising one or more agents capable of restoring intestinal physiological hypoxia.

In certain embodiments, the present invention provides methods for treating GvHD in a subject comprising administering to the subject a therapeutically effective amount of a pharmaceutical composition comprising one or more agents capable of restoring intestinal physiological hypoxia.

In certain embodiments, the present invention provides methods for treating inflammatory bowel disorder in a subject comprising administering to the subject a therapeutically effective amount of a pharmaceutical composition comprising one or more agents capable of restoring intestinal physiological hypoxia.

Such embodiments are not limited to a particular type or kind of subject. In some embodiments, the subject is a human subject. In some embodiments, the subject is a mammalian subject.

Such embodiments are not limited to a specific type or kind of condition associated with impaired intestinal physiological hypoxia. In some embodiments, the condition associated with impaired intestinal physiological hypoxia is intestinal dysbiosis. In some embodiments, the condition associated with impaired intestinal physiological hypoxia is an immune mediated intestinal disease. In some embodiments, the immune mediated intestinal disease is selected from, for example, idiopathic inflammatory bowel disease (IBD), graft versus host disease (GVHD), and enteropathy in primary immunodeficiency. In some embodiments, the condition associated with impaired intestinal physiological hypoxia is related to allogeneic hematopoietic stem cell transplantation (HSCT). In some embodiments, the condition associated with impaired intestinal physiological hypoxia is associated with decreased obligate anaerobes in the subject's microbiome. In some embodiments, the condition associated with impaired intestinal physiological hypoxia is associated with T-cell mediated metabolic defect in intestinal epithelial cells. In embodiments, the condition associated with impaired intestinal physiological hypoxia is related to a dysbiosis inducing event. In some embodiments, the dysbiosis inducing event is treatment with one or more antibiotics (e.g., vancomycin) in connection with surgery. In some embodiments, the dysbiosis inducing event is an infectious disease. In some embodiments, the dysbiosis inducing event is infection by Clostridium difficile. In some embodiments, the dysbiosis inducing event is a primary infection by Clostridium difficile. In some embodiments, the dysbiosis inducing event is a secondary or recurring infection by Clostridium difficile. In some embodiments, the dysbiosis inducing event is traveler's diarrhea.

In some embodiments, the condition associated with impaired intestinal physiological hypoxia is an autoimmune disease, a neurological disorder, diabetes, and/or obesity. Examples of such conditions include, but are not limited to, rheumatoid arthritis, multiple sclerosis diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutieres syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, and inflammatory bowel disease.

Such embodiments are not limited to a specific type or kind of agent capable of restoring intestinal physiological hypoxia. In some embodiments, the agent capable of restoring intestinal physiological hypoxia is an iron binding protein. In some embodiments, the iron binding protein is selected from the group consisting of lactoferrin, transferrin, ferritin, Ferric uptake repressor (FUR) protein, calcineurin, acid phosphatase and ferredoxin. In some embodiments, the iron binding protein is an iron chelator. In some embodiments, the iron chelator is selected from the group consisting of desferrioxamine, Deferasirox, FBS0701, phenanthroline, ethylene diamine tetra-acetic acid (EDTA), diethylene triamine-pentaacetic acid (DTPA) and N,N′-bis[2-hydroxybenzoyl]ethylene diamine-N,N′-diacetic acid (HBED). In some embodiments, the agent capable of restoring intestinal physiological hypoxia is any pharmaceutic agent capable of restoring intestinal eubiosis in the subject.

In some embodiments, administration to the subject of the one or more agents capable of restoring intestinal physiological hypoxia results in a decrease in intestinal oxygen levels in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to baseline intestinal oxygen levels prior to such administering.

In some embodiments, administration to the subject of the one or more agents capable of restoring intestinal physiological hypoxia results in a decrease in abundance of intestinal oxygen in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to baseline intestinal oxygen levels prior to such administering.

Such embodiments are not limited to a particular manner of administering to the subject an agent capable of restoring intestinal physiological hypoxia. In some embodiments, the agent capable of restoring intestinal physiological hypoxia is administered orally. In some embodiments, the agent capable of restoring intestinal physiological hypoxia is administered by oral gavage.

In some embodiments, such methods further comprises administering to the subject one or more of the following additional therapeutic agents: disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors. In some embodiments, the therapeutic agents include, but are not limited to, infliximab, adalimumab, etanercept, parenteral gold or oral gold.

Such methods described herein are not limited to a particular manner of administering the agent capable of agent capable of restoring intestinal physiological hypoxia. In some embodiments, the agent capable of restoring intestinal physiological hypoxia is preferably administered orally (e.g., by oral gavage). However, administration can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, otic, biliary perfusion, cardiac perfusion, priodontal, rectal, spinal subcutaneous, sublingual, topical, intravaginal, transermal, ureteral, or urethral. Dosage forms can be aerosol including metered aerosol, chewable bar, capsule, capsule containing coated pellets, capsule containing delayed release pellets, capsule containing extended release pellets, concentrate, cream, augmented cream, suppository cream, disc, dressing, elixer, emulsion, enema, extended release fiber, extended release film, gas, gel, metered gel, granule, delayed release granule, effervescent granule, chewing gum, implant, inhalant, injectable, injectable lipid complex, injectable liposomes, insert, extended release insert, intrauterine device, jelly, liquid, extended release liquid, lotion, augmented lotion, shampoo lotion, oil, ointment, augmented ointment, paste, pastille, pellet, powder, extended release powder, metered powder, ring, shampoo, soap solution, solution for slush, solution/drops, concentrate solution, gel forming solution/drops, sponge, spray, metered spray, suppository, suspension, suspension/drops, extended release suspension, swab, syrup, tablet, chewable tablet, tablet containing coated particles, delayed release tablet, dispersible tablet, effervescent tablet, extended release tablet, orally disintegrating tablet, tampon, tape or troche/lozenge.

Intraocular administration can include administration by injection including intravitreal injection, by eyedrops and by trans-scleral delivery.

Administration can also be by inclusion in the diet of the mammal such as in a functional food for humans or companion animals.

As noted, it is preferable that such agents agent capable of restoring intestinal physiological hypoxia are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methylcellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated such as to provide rapid, sustained, or delayed release of the active ingredients after administration to the patient by employing procedures well known in the art. The formulations can also contain substances that diminish proteolytic degradation and promote absorption such as, for example, surface-active agents.

The specific dose can be calculated according to the approximate body weight or body surface area of the patient or the volume of body space to be occupied. The dose will also depend upon the particular route of administration selected. Further refinement of the calculations necessary to determine the appropriate dosage for treatment is routinely made by those of ordinary skill in the art. Such calculations can be made without undue experimentation by one skilled in the art in light of the activity in assay preparations such as has been described elsewhere for certain compounds (see for example, Howitz et al., Nature 425:191-196, 2003 and supplementary information that accompanies the paper). Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

The present invention also provides kits comprising an agent capable of restoring intestinal physiological hypoxia and other therapeutic agents.

### EXPERIMENTAL

The following examples are provided to demonstrate and further illustrate certain preferred embodiments of the present invention and are not to be construed as limiting the scope thereof. As used herein, personal pronouns such as “our”, “we”, “I”, etc. refer to the inventors of the present invention.

### Example I

This example demonstrates that allogeneic dysbiosis is not pathogenic to naïve animals.

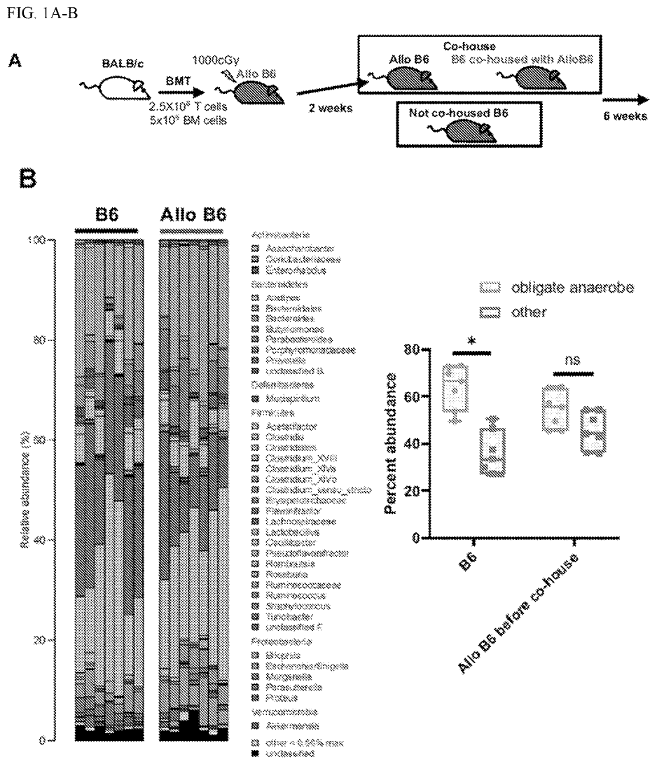

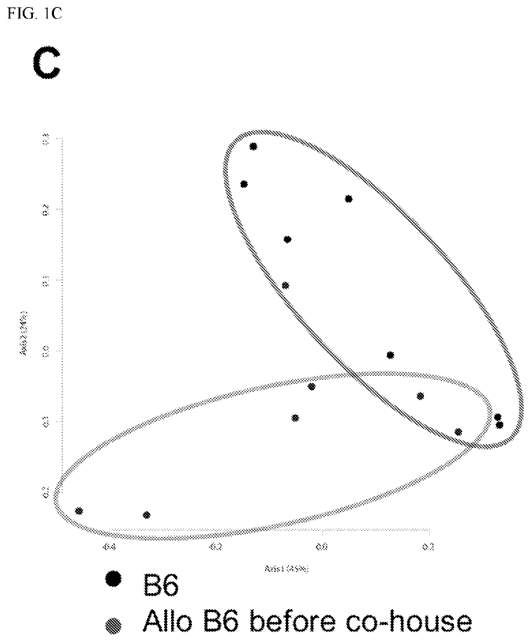

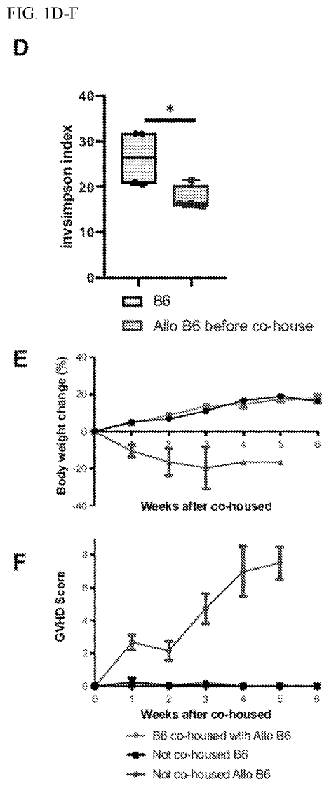

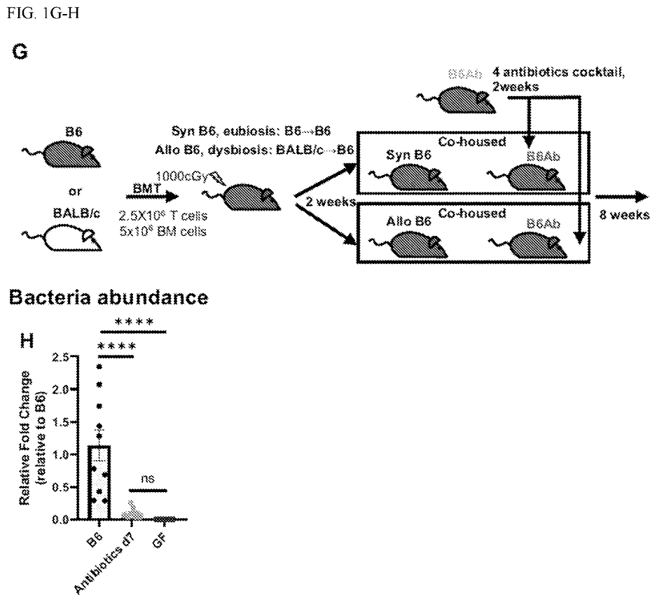

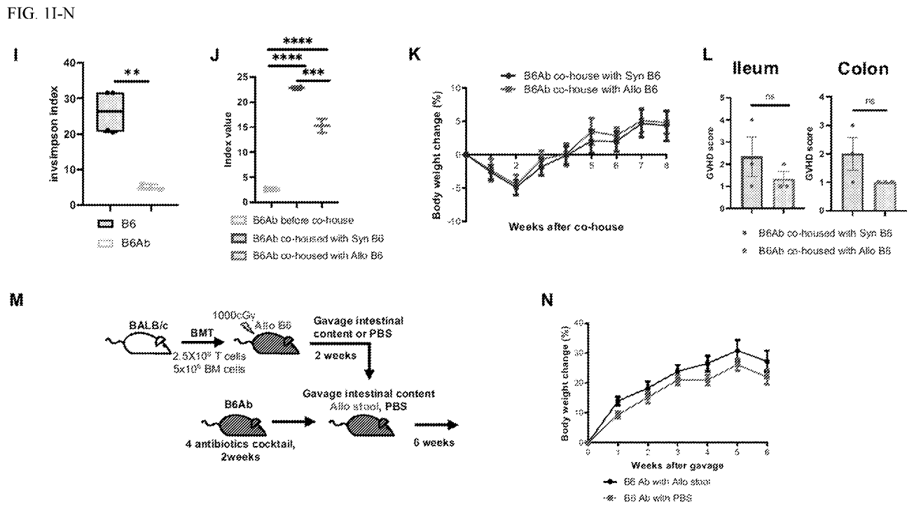

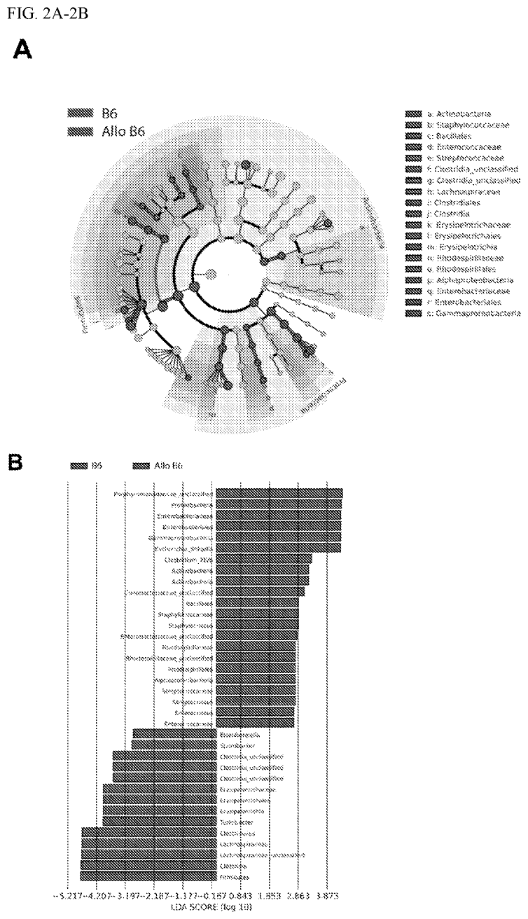

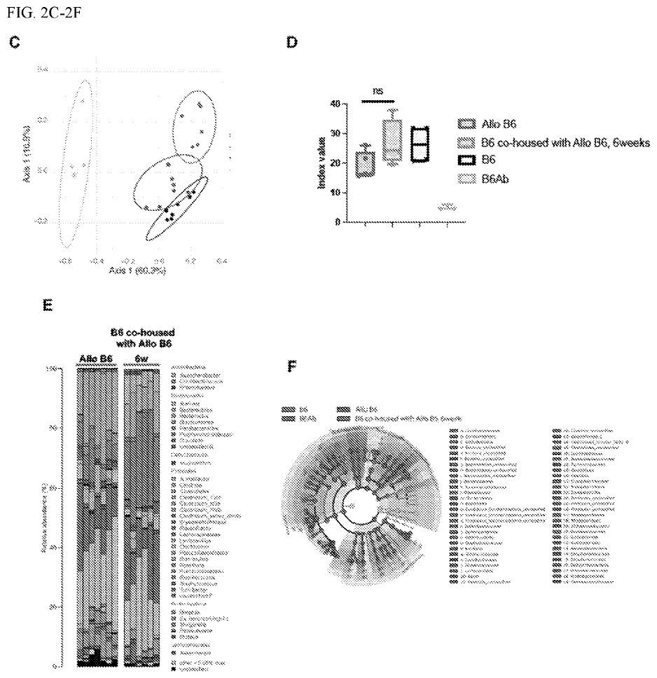

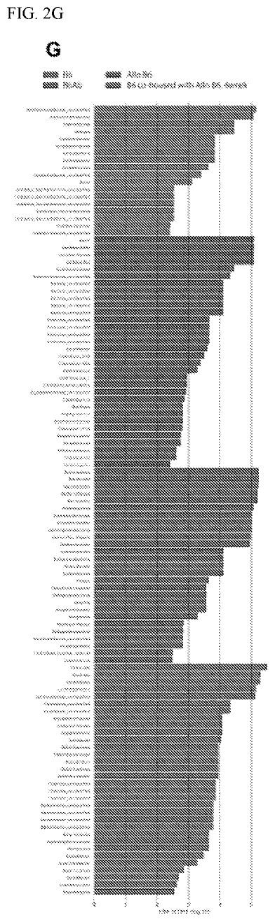

We first determined whether dysbiosis associated with GVHD following allogeneic HSCT is toxic in the absence of HSCT. Naïve C57BL/6 (B6) animals were irradiated with 10 Gy and transplanted with splenic T cells and TCD BM from allogeneic BALB/c donors (Allo B6) to induce GVHD and dysbiosis (FIG. 1A). Dysbiosis in stool from Allo B6 animals was confirmed at 2 weeks (FIG. 1B to D, FIGS. 2A and B). Allo B6 stools contain more Proteobacteria, particularly facultative anaerobes in the Enterobacteriaceae family such as Escherichia (FIGS. 1A and B). Allo B6 animals were then cohoused together with a cohort of naïve, un-transplanted B6 hosts for 6 weeks (B6 co-housed with Allo B6), while a cohort of naïve B6 animals from the same colony were not co-housed (Not co-housed B6) and served as control animals (see FIG. 1A). Microbiome of B6 co-housed with Allo B6 changed towards Allo B6 at 6 weeks (FIG. 2C to G). Despite the shift in community structure and reduction in diversity, both the cohoused and non-cohoused cohorts of un-transplanted naïve B6 mice showed similar weight gain with no signs of GVHD (FIGS. 1E and F).

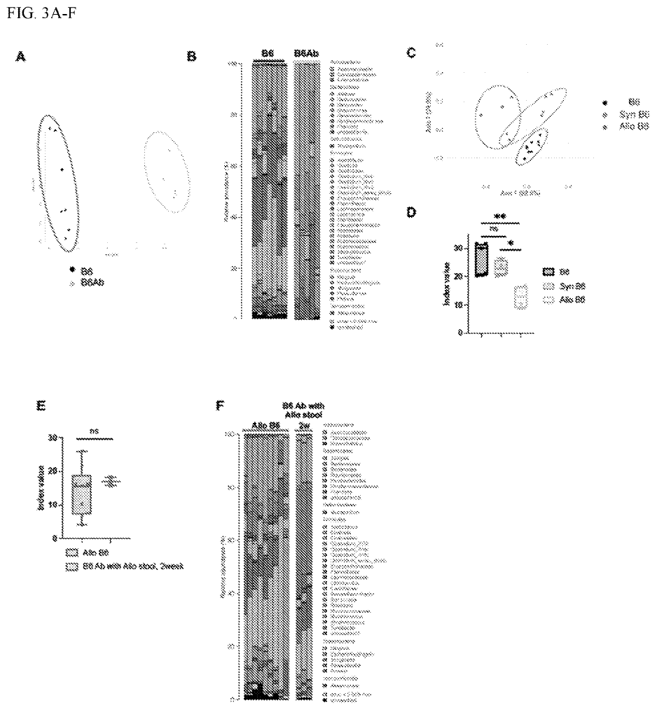

It is possible that some organisms in the endogenous normal microbiome of the cohoused mice may have prevented the pathogenic effects of dysbiosis. Therefore, we next examined the impact of dysbiosis in naïve B6 animals that were treated with a cocktail of four antibiotics that have been shown to effectively clear endogenous GI microbiome in B6 hosts³. The naïve B6 animals were treated with the four-antibiotic cocktail for 2 weeks (B6Ab, FIG. 1G), and near complete loss of microbiome was confirmed (FIGS. 1H and I, FIGS. 3A and B) before cohousing with Allo B6 as above. To further control for the ability of microbiota from transplanted mice to take colonize naïve antibiotic treated B6 hosts, we also cohoused another cohort of these animals with B6 syngeneic BMT (Syn B6, FIG. 1G). We confirmed that B6 and Syn B6 showed similar microbiome and eubiosis (FIGS. 3C and D). The naive, un-transplanted B6 animals were cohoused and monitored for 8 weeks for signs of weight loss and GI damage. Co-housing with Allo B6 caused dysbiosis (FIG. 1J). As shown in FIGS. 1K and L, cohousing of the un-transplanted antibiotic treated naïve B6 hosts with either dysbiotic Allo B6 or the control Syn B6 animals did not impact weight gain and not cause the GVHD.

It is formally possible that cohousing alone might not be sufficient to cause complete microbiota alterations and cause dysbiosis in un-transplanted naïve B6 animals despite pre-treatment with antibiotic cocktails. Therefore, to mitigate any potential inefficiencies of cohousing on changes to the microbiome, we treated the naïve B6 un-transplanted hosts with the antibiotics or diluent control as above and then directly gavaged them with intestinal content from Allo B6 animals (FIG. 1M) and monitored as above. Gavage of antibiotic treated naïve B6 animals with Allo B6 stools caused dysbiosis (FIGS. 3E and F). Un-transplanted antibiotic treated naïve B6 animals that received dysbiotic stool gavage from Allo B6 animals showed similar body weight gain as control un-transplanted antibiotic treated naïve B6 controls that received PBS gavage (FIG. 1N).

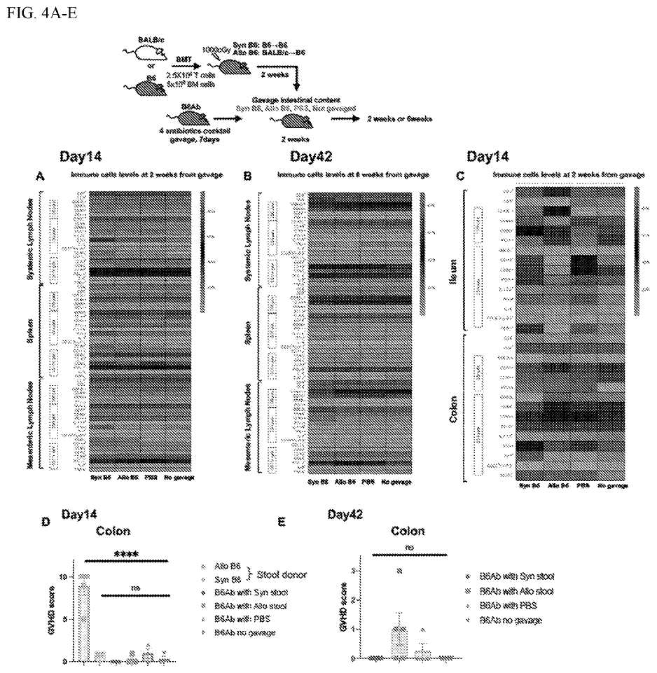

While clinical features of GI damage were not apparent, it is possible that gavage of the dysbiotic stool into the antibiotic treated, naïve un-transplanted B6 mice might cause changes in host immunity. We therefore gavaged Allo B6 stool, the control Syn B6 stool, PBS, or sham into antibiotic treated animals as above and performed systematic phenotyping of T cells (phenotype, cytokine, regulatory T cells) and dendritic cells in distant secondary lymph nodes (LN), spleen and regional (mesenteric) LNs on days 14 and 42 days after the stool gavage (FIGS. 4A and B). No significant differences were noted the in the CD4+ and CD8+ subsets, PDT, CD69, 62L, 44, CD4++Foxp3 Tregs, T cell IFNγ, IL-4 and IL-17A, or the CD8+0, 86, 40, PDL1, IL-6 and TNFα expression in the CD11c⁺ cells) between the cohorts. Furthermore, the local tissue T cell phenotype (in colon and ileum) and numbers were also similar between all of the cohorts (FIG. 4C).

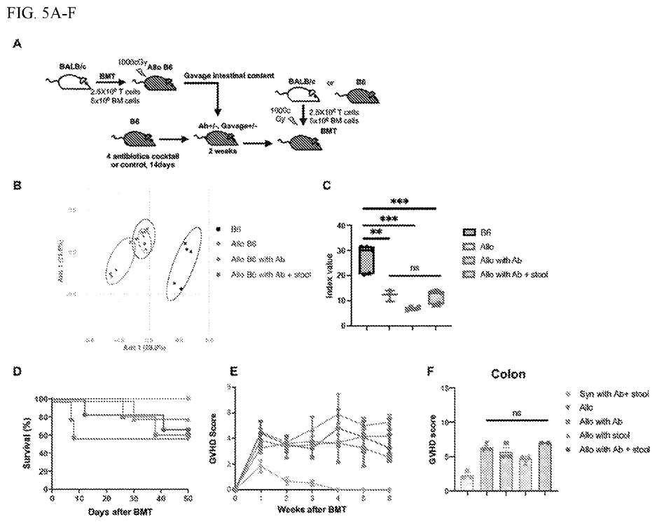

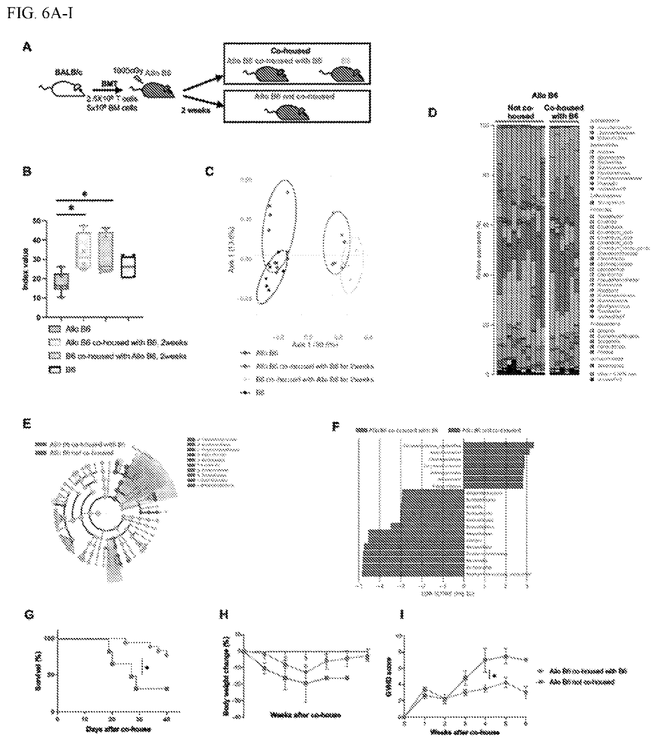

Furthermore, when we analyzed histopathological changes in the small and large intestines that may have been caused by transfer of dysbiotic stool, we found no evidence of damage or significant differences between the groups at 2 weeks (FIG. 4D) or 6 weeks (FIG. 4E) after intestinal content gavage. These data thus collectively demonstrate that a shift towards dysbiosis did not affect the intestinal health of the un-transplanted naïve B6 animals and that allo-dysbiosis by itself is not deleterious in the absence of preceding damage. Thus, dysbiosis after allo-HCT is not a cause, but is a consequence of GVHD.

### Example II

This example demonstrates that post-transplant but not pre-transplant dysbiosis regulates GVHD.

We next addressed whether the changes to the microbiome after allo-HCT amplify or attenuate GVHD severity. To this end, we first explored whether host dysbiosis before transplant has an impact on the outcomes of GVHD. B6 animals were treated with either antibiotics or diluent controls and then reconstituted with stool microbiome from Allo B6 animals (FIG. 5A). They were then lethally irradiated (10 Gy) and transplanted with 5×10⁶ BM cells and 2.5×10⁶ splenic T cells from either B6 syngeneic or allogeneic BALB/donors. Allogeneic recipients with or without antibioticis and stool gavage showed dysbiosis and similar microbiome between allogeneic groups, but changes from B6 mice before HCT (FIGS. 5B and C). Surprisingly, all of the allogeneic recipients with or without dysbiosis prior to the transplant showed similar mortality and clinical GVHD severity (FIGS. 5D and E). Histopathology of the intestine confirmed similar pathological GVHD severity (FIG. 5F). These data suggest that the status of the pre-transplant microbiome structure, specifically neither dysbiosis nor eubiosis, has significant impact on GVHD outcomes.

Pretransplant dysbiosis does not address the issue of the impact of the change from pre-BMT eubiosis to post-BMT dysbiosis. Because GVHD is always associated with post-transplant dysbiosis, we examined whether prevention of post-transplant dysbiosis would mitigate GI GVHD (FIG. 6A). B6 animals received allogeneic BMT described in Methods. Each individual recipient mouse was cohoused with an individual naïve B6 WT mouse in a single cage from day 14 after HSCT. Simpson diversity and PCoA analyses of stools from these recipient mice revealed that cohoused allogeneic mice (Allo B6 co-housed with B6 for 2 weeks) had higher diversity and different microbiota compositions from single housed allogeneic mice (Allo B6 not co-housed) (FIG. 6B to F) demonstrating that the cohousing shifted the stool microbiome of Allo B6 recipients towards the healthy microbiome. Notably co-housed Allo B6 recipients demonstrated greater survival, reduced weight loss and milder clinical GVHD than not co-housed Allo B6 (FIG. 6G to I). Taken together with above, the data demonstrate that dysbiosis pre-transplant per se are not pathogenic, i.e., disease enhancing, but that promoting post-BMT eubiosis ameliorates the severity of GVHD.

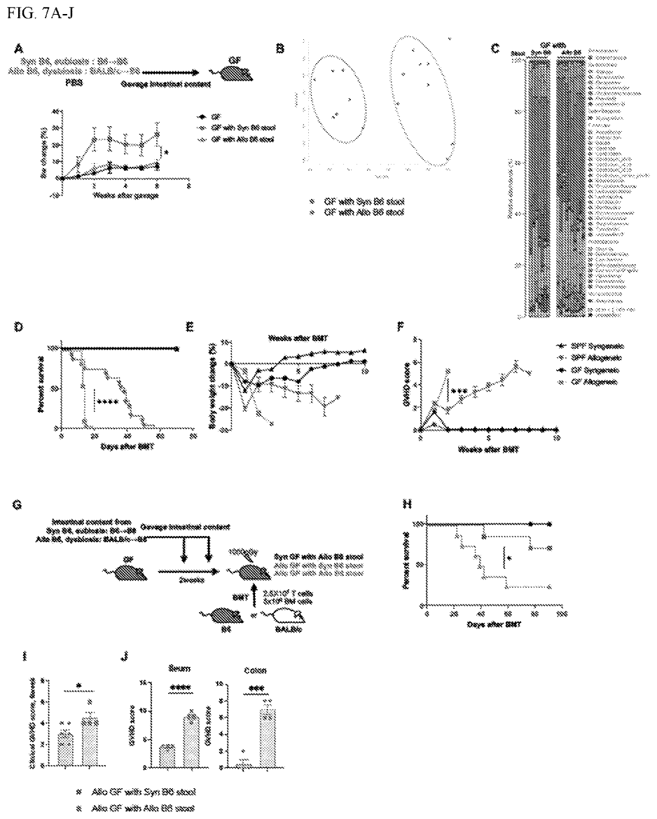

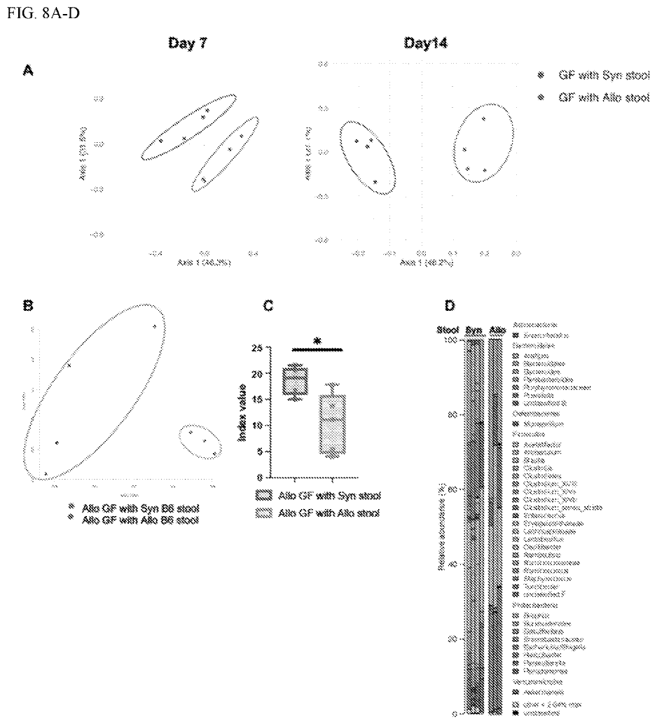

It is possible that the above effects could be secondary to pre-existing microbiota or from the experimental effects of gavage and antibiotic use. Therefore, to definitively determine whether dysbiotic stool has any intrinsic pathogenic effects and eubiosis has beneficial effects in the absence of above variables, we next compared weight gain of GF mice with littermate GF mice that were conventionalized with either dysbiotic allo-stool or eubiotic syngeneic stool (FIG. 7A). GF animals that received eubiotic stool from syngeneic mice and those that were conventionalized with dysbiotic allo-stools demonstrated weiaght gain when compared with GF controls confirming that dysbiotic microbiome by itself is not pathogenic in the absence of prior damage (FIG. 7A, 7B, C and FIG. 8A).

### Example III

This example demonstrates that absence of host microbiome aggravates GVHD.

Our data demonstrate that changes in endogenous GI microbiome structure after allo-HCT are not a cause but a consequence of GVHD and suggest that they might play a salutary role as opposed to a pathogenic role after BMT^{3 4 16 22 23}. However, before the advent of modern stringent germ-free (GF) facilities, and before technologies to confirm presence of microbes such as 16S sequencing, led to the paradigm that GF mice show reduced GVHD¹⁰, suggesting an overall pathogenic role for host microbiome. Therefore, to elucidate the importance of microbiota definitively, and directly, in the gut before BMT, we next hypothesized that in contrast to the current paradigm, GF recipients will demonstrate greater GVHD than normal SPF animals. To rule out any potential confounding littermate effects at birth between GF and SPF conditions, age-matched GF B6 littermate mice were first separated into two cohorts at 4-6 weeks of age. One group was continued under stringent GF conditions and the second group was aged under SPF conditions up to age 12-14 weeks. These GF and SPF cohorts were then irradiated and transplanted with either syngeneic B6 or allogeneic BALB/c donors and were followed post-BMT under stringent GF or SPF conditions respectively. All the syngeneic recipients from both the GF and SPF cohorts survived and showed no signs of GVHD demonstrating that absence of microbiome did not cause mortality in the absence of allo-reactive T cell mediated damage. All of the allogeneic recipients under SPF conditions, as expected, demonstrated signs and severity of GVHD with a median survival of 7 weeks (FIG. 7D to F). However, in contrast to the existing paradigm, the GF mice that received allogeneic HSCT demonstrated significantly greater mortality, weight loss and clinical GVHD (FIG. 7D to F).

Because allo-stool (dysbiotic microbiome) neither caused disease nor improved weight gain in GF mice while eubiotic microbiome improved weight gain in GF animals, we next surmised that GF mice transplanted with healthy microbiome will show reduced GVHD. To this end GF animals received allo-stool or syngeneic stool (FIG. 7G). The engraftment and fidelity of the transplanted microbiome was confirmed (FIG. 8B to D). These animals were then irradiated and utilized as recipients of either syngeneic or allogeneic BMT as in Methods (FIG. 7G). Syngeneic recipients that received dysbiotic microbiome survived, demonstrating, once again, no inherent toxicity from the dysbiotic stool. The allogeneic recipients of dysbiotic stool demonstrated expected GVHD mortality and clinical/pathological GVHD (FIG. 7H to J). By contrast allo-recipients that were transplanted with eubiotic healthy microbiome demonstrated significantly greater survival and reduced weight loss and clinical/pathological GVHD (FIG. 7H to J). Thus, the structure of the host microbiome is dispensable for induction of GVHD, but is critical for regulation of GVHD after it has been initiated.

### Example IV

This example demonstrates that loss of intestinal physiologic hypoxia causes dysbiosis.

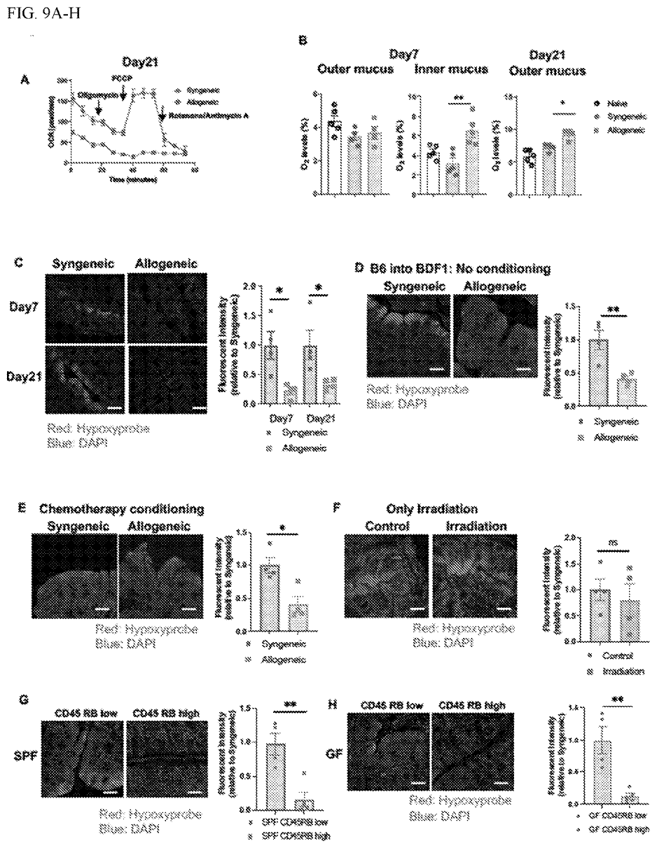

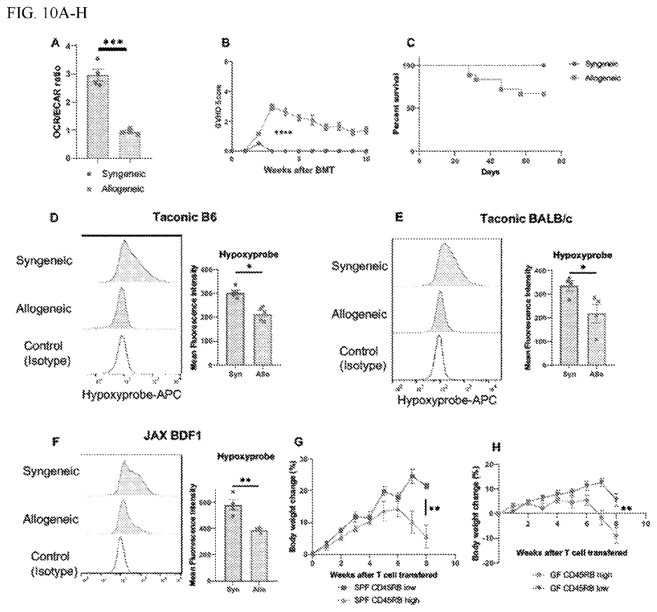

We next explored the mechanisms for development of dysbiosis following allo-BMT. The physiologic hypoxia (low oxygen) present at homeostasis in the intestinal milieu regulates the composition and structure of the intestinal microbiome¹. In the context allo-BMT, host IECs demonstrate a metabolic defect in OXPHOS due to a T cell induced disruption of mitochondrial complex II²¹. We therefore hypothesized that lack of utilization of cellular O₂ caused by disruption of OXPHOS in the IECs after allo-HCT will lead to loss of physiologic hypoxia (increase in O₂) and thus cause dysbiosis by making the GI tract more permissive for growth of aerotolerant and facultative anaerobes at the expense of obligate anaerobes. To this end we systematically measured oxygen consumption of the IECs, the changes in the actual intestinal luminal, intestinal mucosal levels of O₂ by colonoscopy, and intestinal cellular hypoxia by pimonidazole after HSCT. B6 received either syngeneic B6 or allogeneic BALB/c transplants as in Methods. Consistent with previous report, CD326⁺ IECs harvested from the allogeneic mice on day 21 after HSCT demonstrated a significant reduction in oxygen consumption rate (OCR) when compared with IECs from syngeneic controls (FIG. 9A). Furthermore, IECs from allogeneic animals did not respond to treatment with carbonyl cyanide-p-trifluoromethoxyphenylhydrazone (FCCP), a mitochondrial uncoupler, when compared with IECs from syngeneic animals demonstrating the reduction in allo-IEC mitochondrial ETC functions and utilization of O₂ (FIG. 9A). However, both syngeneic and allogeneic IECs showed similar extracellular acidification rates (ECAR, an indicator of glycolysis), with significantly reduced OCR/ECAR ratio (FIG. 10A) demonstrating that the defect in O₂ utilization should increase O₂ levels leading to a loss of intestinal luminal and cellular physiological hypoxia.

To confirm increase in O₂, we performed colonoscopy with probes that directly measure oxygen levels in the colonic mucosa (just adjacent to IECs) and in the center the intestinal lumen of the HSCT recipients. The O₂ concentrations in deeper locations, in the inner mucus of the colon, immediately adjacent and closer to the IECs was greater in the allo-recipients than the controls (FIG. 9B). The oxygen levels in the lumen, further away from the IECs initially demonstrated similar levels of oxygen in allo-recipients as in the controls naïve or syngeneic B6 controls early after BMT (FIG. 9B). However, 3 weeks after BMT, the allo-recipients demonstrated significantly greater levels of O₂ in the intestinal lumen when compared with the controls demonstrating a loss of hypoxia in the lumen farther away from the IECs (FIG. 9B). Thus, both colonic and luminal O₂ levels were increased in allo-HSCT animals. Finally, loss of intestinal cellular hypoxia with pimonidazole was also documented in the allo-BMT recipients at days 7 and 21 after HSCT (FIG. 9C). Thus, underutilization of the O₂ by the IECs because of T cell induced metabolic defect leads to loss of intestinal physiological hypoxia in GVHD.

Next, to validate whether the loss of physiological intestinal hypoxia is exclusively from T cell mediated GVHD and not a consequence of strain dependent artifact, or damage from conditioning we determined the status of intestinal hypoxia in the non-irradiated parent into F1 model (B6→B6D2F1), where alloreactive donor T cells cause GVHD despite absence of any conditioning (FIGS. 10B and C). Allogeneic IECs demonstrated significantly increased levels of oxygen (FIG. 9D). Similar loss of hypoxia was also observed when allogeneic B6 animals were conditioned with chemotherapy (busulfan and cyclophosphamide) prior to HSCT (FIG. 9E). By contrast, when animals were conditioned with irradiation alone there was no loss of hypoxia (FIG. 9F). To demonstrate that the loss of physiological hypoxia is independent in differences of microbiome composition, we utilized mouse strains from a different mouse supplier (Taconic, JAX). Allogeneic Taconic B6, Taconic BALB/c and JAX BDF1 recipients showed similar loss of hypoxia at day 7 after BMT (FIG. 10D to F). These results extend previous observations and demonstrate that the increase in O₂ concentration in the intestines is observed only in the context of allo-T cell mediated damage of GI tract²¹. To further determine whether this can be extended to other T cell mediated damage of GI tract, we also determined intestinal oxygen concentration in the CD45RB^{hi} adoptive T cell mediated autoimmune model of IBD. Induction of T cell mediated autoimmune colitis also led to loss of hypoxia in both SPF (FIG. 9G, FIG. 10G) and GF animals (FIG. 9H, FIG. 10H). Thus, T cell mediated disruption of intestinal cell OXPHOS in the IECs leads to poor utilization of cellular O₂ resulting in loss of physiologic hypoxia that promotes an environment permissive for dysbiosis.

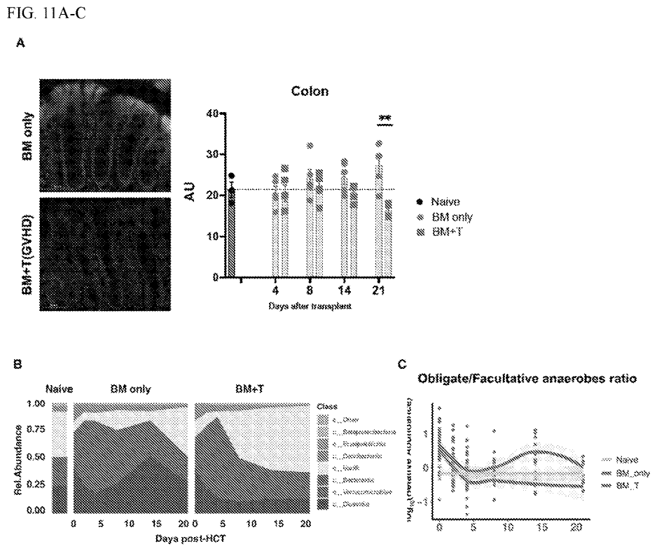

Next, to enhance the generalizability and to assess whether loss of physiological hypoxia is not an artifact of the specific vivarium conditions, we performed the similar experiments with different strain combination at a second institution (MSKCC, NY Center). Similar to the data in University of Michigan, HCT recipients with GVHD showed loss of hypoxia in colon (FIG. 11A). The changes of microbiome composition (FIG. 11B) and the decreased ratio of obligate/facultative anaerobes in recipients with GVHD (FIG. 11C) were observed. These data demonstrated GVHD induced loss of hypoxia and dysbiosis in colon with independent of the institution, vendors and strains.

### Example V

This example demonstrates intestinal O₂ levels regulate GVHD in a microbiome dependent and independent manner.

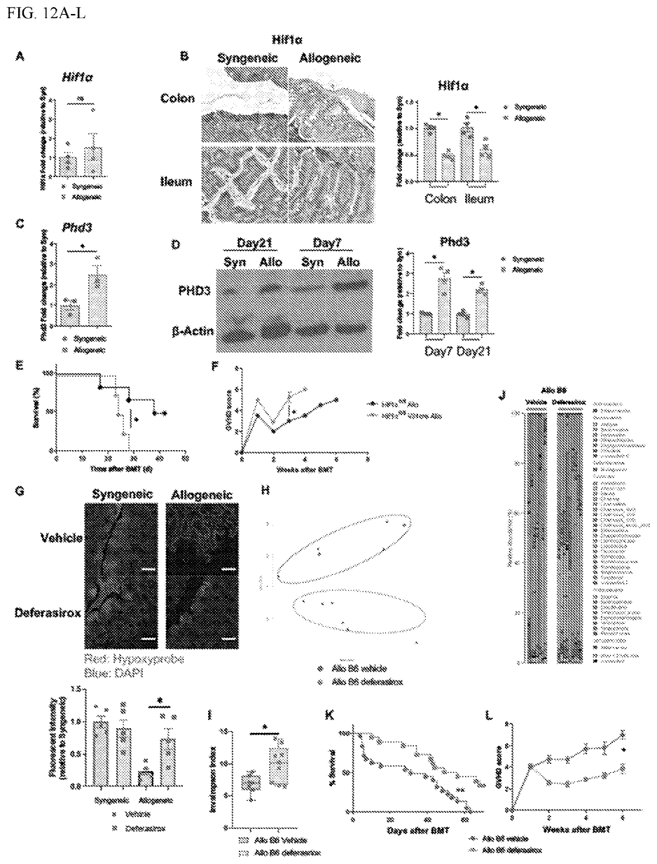

We next determined whether the loss of physiologic hypoxia has direct effect on IECs that could impact GI GVHD. To analyze the impact of increase in O₂ we determined the expression of cellular O₂ sensor, hypoxia inducible factor (HIF-1α). B6 animals received syngeneic or allogeneic BMT as in Methods. Hif-1α gene expression was similar in the IECs harvested from allogeneic and syngeneic animals (FIG. 12A). However, the HIF-1α protein was lower in the IECs from allogeneic animals when compared to syngeneic animals on day 21 after BMT (FIG. 12B). By contrast, the expression of proryl hydroxylase 3 (PHD3), a protease that regulate the protein level of HIF-1α, was upregulated in the allo-IECs (FIGS. 12C and D).

Next, to determine whether the IEC cell autonomous of the loss of physiologic hypoxia after allo-HSCT has a microbiome independent effect on GI GVHD, we utilized littermate congenic IEC specific Hif-1a gene knock out mice (Hif1a^{fl/fl} Villin1cre B6) or the WT Hif1a^{fl/fl} B6 animals (to harmonize pre-HCT microbiome) as allogeneic HSCT recipients. The animals were also cohoused post-BMT to harmonize microbiome changes. The TEC specific Hif1a^{fl/fl} Vil1cre B6 allogeneic recipients showed significantly greater severity of GVHD and mortality when compared to the littermate Hif1a^{fl/fl} B6 mice despite being cohoused together (FIGS. 12E and F). Taken together these data demonstrate that changes in O₂ after allo-HSCT has direct effects host IECs that impacts GVHD severity independent of changes in the microbiome.

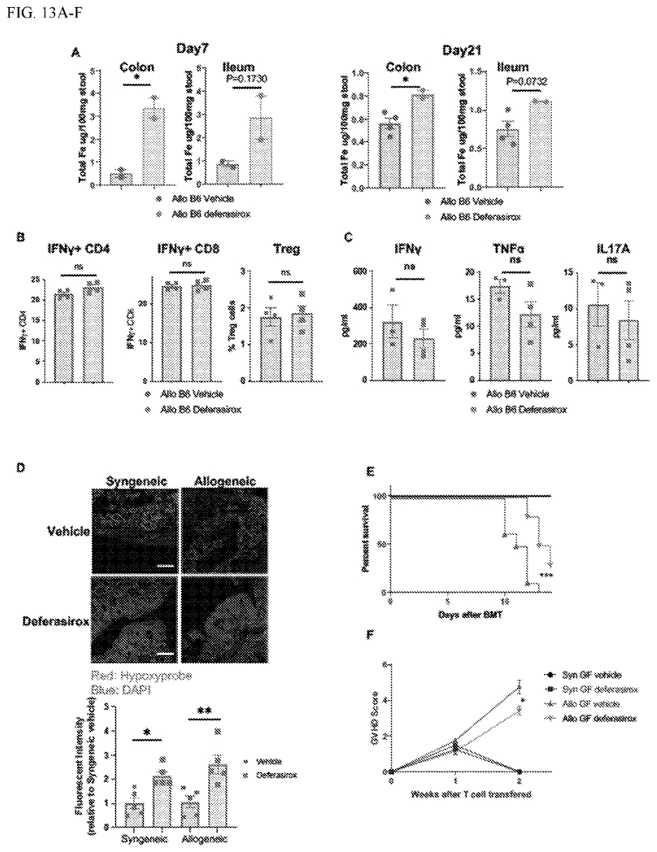

We next determined the direct functional link between the increase in O₂, the loss of physiologic hypoxia, observed after allo-HSCT to the resultant dysbiosis and GVHD severity. To mitigate the impact of excess luminal O₂ by reducing the Fenton reaction in the IECs, we administered the iron chelator, deferasirox, by oral gavage after BMT as in Methods^{24 25}. We determined the experimental dose and timing by measuring the level of iron in the stool from both small and large intestines following gavage with Fe chelator (FIG. 13A). We first determined whether gavaging with deferasirox altered the level of O₂ concentration in the intestines after HSCT and found that it mitigated loss of hypoxia in the allogeneic recipients (FIG. 12G). The PCoA analysis showed different microbial composition between vehicle and deferasirox treated allogeneic animals (FIG. 12H), with an increase in microbial diversity (FIGS. 12I and J) thus demonstrating that changes in O₂ directly contributed to dysbiosis after allo-HSCT. We next hypothesized that squelching of O₂ that lead to amelioration of dysbiosis after allo-HSCT will lead to attenuation of GVHD mortality. Consistent with the hypothesis, deferasirox treated allogeneic animals demonstrated significantly reduced severity of clinical GVHD and survival (FIGS. 12K and L). To explore the iron chelator effects on T cells, we determined the changes of T cell phenotype in recipients. The iron chelator did not show any effect on inflammatory cytokine secretion and Treg ratio in spleen (FIGS. 13B and C).

Because alteration of O₂ affects host IECs in addition to the microbiome, we next determined whether restoring intestinal hypoxia by Fe chelation after allo-HSCT mitigated GVHD independent of its impact on dysbiosis. To determine microbiome independent effects, we utilized GF B6 animals as allo-HSCT recipients and treated them with deferasirox or diluent control (see Methods). All transplanted mice were also maintained in stringent GF environment post BMT. Treatment with Fe chelation also ameliorated loss of hypoxia after BMT in the GF recipients (FIG. 13D). All of the syngeneic GF mice treated with either diluent control or deferasirox survived without GVHD. The allogeneic GF mice treated with diluent control demonstrated severe GVHD and died within 2 weeks after BMT (FIGS. 13E and F). By contrast, deferasirox treated allogeneic GF mice (FIGS. 13E and F) demonstrated improved survival when compared with diluent treated allo-recipients, but eventually succumbed to GVHD. These data collectively demonstrate that O₂ modulation, to improve intestinal physiologic hypoxia has direct salutary effects both on host IECs and the host microbiome to improve GI GVHD.

### Example VI

This example provides a discussion related to Examples I-V.

Imbalance in colonic microbiome, termed dysbiosis, is associated with human intestinal diseases, including GI GVHD^{1 9}. However, several key questions remain. Specifically, whether the change in microbiome structure after allo-HCT is a cause or consequence, an amplifier or a mollifier of GVHD, the mechanisms that cause dysbiosis and if those mechanisms can be targeted to mitigate GVHD remain elusive. Herein we address these gaps and demonstrate that pre-transplant dysbiosis by itself is not pathogenic, and that post-HCT dysbiosis is a consequence of the changes in intestinal luminal oxygen level from the tissue injury caused by allogeneic donor T cells after allo-HCT. We additionally demonstrate that correction of post-transplant dysbiosis has beneficial impact. We show that in contrast to paradigm that was established several decades ago, absence of microbiome after HCT aggravates GVHD, and thus provide clarity to the emerging data that the structure of the microbiome after HCT, regulates the severity of GI GVHD^{3 10 16 17 21 22}. These data extend previous observations that T cell mediated disruption of intestinal OXPHOS leads to the loss of luminal physiological hypoxia (increase in O₂)²¹, which functions as a control switch to shift the intestinal microbiome from predominantly obligate anaerobes to facultative anaerobes and aerotolerant microbes, thus providing mechanistic insights into the cause of dysbiosis. Finally, targeting excess O₂ with iron chelation promoted physiologic hypoxia, mitigated dysbiosis and attenuated GVHD severity, thus suggesting that Fe chelation with available oral drugs may be a novel strategy to clinically mitigate GVHD severity.

During homeostasis, the host intestinal epithelial cell metabolism is dependent on oxidative phosphorylation, resulting in high epithelial oxygen consumption and as a consequence epithelial and luminal hypoxia¹. Epithelial hypoxia helps to maintain a microbial community dominated by obligate anaerobic bacteria at homeostasis^{1 20}. We build on prior observation that the disruption of host IEC metabolism pathogenic T effector cells results in an increase in intestinal oxygen, thereby driving an expansion of facultative anaerobic and aerotolerant bacteria, a hallmark of dysbiosis GVHD^{4 22 23}. Thus, one critical mechanism for dysbiosis, the loss of physiologic hypoxia in a non-infectious intestinal diseases like GVHD and IBD are similar to that observed following infection by enteric infectious pathogens^{1 22}. This shared mechanism linked to tissue oxygenation in causing dysbiosis allows for a better understanding of shifts in microbial ecosystem may also be germane to other diseases associated with dysbiosis. Nonetheless, it is possible that the dominant mechanisms may be distinct in other disorders, particularly in the context of dysbiosis associated with non-enteric diseases.

Seminal studies performed many years ago suggested that germ-free hosts had less severe GVHD^{9 10}. These studies formed a paradigm for the field of HCT, even as they appeared to stand in contrast to emerging data that demonstrate strong correlation between microbiome and GVHD. The modern-day studies, however, showing shifts in microbiome or use of antibiotics as potential regulators of GVHD, do not address the notion of stringent germ-free status on GVHD. We evaluated the role of stringent germ-free status on GVHD and found that complete absence of microbiome in transplanted mice that are housed in germ-free conditions show greater severity and mortality from GVHD than normal SPF recipients. These data directly demonstrate an overall salutary role for microbiome in GVHD. The reason for the different results from the studies by van Bekkum et al, is likely because those earlier studies were performed in an era when sequencing for microbiome was not yet available and as such the ability to confirm true germ-free status or the reliability of the colony conditions to be maintained in a germ-free state was likely not optimal^{10 26}. Furthermore, our data demonstrate that adding back enteric microbiome mitigated GVHD severity in GF hosts, thus definitively demonstrating a critical role for microbiome in regulation of GVHD.

Our data utilizing cohousing SPF, antibiotic treated, and GF animals provide several insights into the role of dysbiosis before and after allo-HCT. First, dysbiosis by itself, in the absence of initial damage by alloreactive T cells, is not pathogenic, at least in the period that acute GVHD mortality typically occurs in these systems. Second, the presence of dysbiosis prior to HCT did not aggravate GVHD. However, promotion of eubiosis after HCT mitigated GVHD. Similarly, GF mice conventionalized with healthy stools mitigated GVHD. These data, when taken together with observations that outcomes are worse in humans with use of antibiotics that primarily target anaerobes after HCT demonstrate that microbiome primarily has a salutary role in GVHD. Thus, promotion of eubiosis after HCT (i.e prebiotic or probiotic approaches with appropriate antibiotic stewardship for growth of healthy microbiome, i.e., obligate anaerobe microbes) as opposed to treating dysbiosis before (eliminating facultative anaerobes or aerobes, i.e. only antibiotic approaches) may be a rational strategy to therapeutically modulate microbiome to ameliorate GVHD. The mechanisms by which eubiosis after HCT may mitigate GVHD is likely (but not limited) to generation of metabolites by obligate aerobeic commensals that nourish the IECs, or promote immune and tissue tolerance, or provide nutrients to healthy commensal and also regulate the microbial ecology by promoting a milieu that is not permissive for pathobionts^{16 17 21 27 28 29 30}. However, the biology of GVHD is complex with several studies demonstrating a role for DAMPs and PAMP production by tissue damage as amplifiers of a GVH response^{31 32}. Therefore it is possible that dysbiosis when characterized by expansion of pathobionts that gain access to hosts circulation and tissues amplifies GVHD^{4 22 33}.

The increase in, and the type of pre-transplant dysbiosis noted in the clinical studies that correlated with outcomes suggest that factors that lead to dysbiosis could predict worse GVHD, but do not directly demonstrate that dysbiosis by itself directly caused GVHD. Nonetheless, it is possible, even if unlikely based on our GF reconstitution experiments, that there might be a threshold for the specific type, or degree of dysbiosis, beyond which pre-transplant dysbiosis may cause or promote GVHD. The change in specific bacteria (Enterococcus and reduction in Blautia), has been hard compare the different studies, but nonetheless all of the studies are broadly consistent with reduction in obligate anaerobes and an increase in proportion of aerotolerant microbiome following GVHD. Thus the nature of specific microbes and their impact in the context of the wider ecology will need to be assessed in future studies with GF mice monocolonized or polycolonized with depletion of specific microbe of interest to directly assess their pathogenic or beneficial effects, alone, and in the context of other microbes. However, our observations, regarding hypoxia loss in GVHD and development of dysbiosis are consistent across vendor source, different strains, multiple model systems and under different experimental conditions from different institutions.

It remains unknown whether the degree of GVHD severity affects microbiome contents in a specific manner. However, our data, consistent with previous data, have shown that there is a general loss of diversity. It remains to be determined whether the measurement like the specific and absolute loss of diversity can be consistently quantified and compared across different patients and contexts, and to formally assess and rule out that the degree of dysbiosis may directly cause GVHD. Additionally, the improvement of survival in co-housing experiments could be confounded by the social, behavioral or neurological effects of cohabitation, besides, or in addition to, changes in microbiome. These potential variables will need to be investigated in carefully designed future studies.

During homeostasis, despite being in a relative hypoxic environment, intestinal metabolism is directed toward oxidative phosphorylation (OXPHOS), resulting in high epithelial oxygen consumption. Disruption of OXPHOS in the IECs by alloreactive T cells, reduces oxygen consumption²². Our data now extends these observations and provides a potential novel therapeutic approach. It demonstrates that increase in luminal oxygen makes the intestinal environment less permissive for obligate anaerobes, thus promoting dysbiosis. The excess tissue oxygen levels is also corroborated by lower expression of the O₂ sensor HIF-1α in the IECs^{34 35 36}. It is likely that the lack of O₂ consumption might also contribute by direct free radical injury to the IECs and increase GVHD severity. Administration of oral iron chelator reduced excess luminal O₂, promoted physiological hypoxia, improved microbial diversity, reduced dysbiosis and ameliorated GI GVHD, but did not alter donor T cell immune responses in the host. Moreover, oral Fe chelation also modestly improved GVHD survival in GF animals demonstrating microbiome independent effects. Thus, squelching of excess O₂ by Fe chelation improved GI GVHD largely in a microbiome dependent, but also, to a modest degree, in a microbiome independent manner. The specific mechanisms for microbiome independent effects of Fe chelation on host tissues, such as ferroptosis, will need further investigation^{37 38}. Furthermore, Fe chelation likely impacts Fe availability to microbes directly affecting the structure of microbiome³⁹. Nonetheless, our data thus provide a novel role for tissue O₂ as a mechanism for dysbiosis and Fe chelation as a therapeutic target for amelioration of non-infectious immune mediated intestinal diseases.

### Example VII

This example provides the materials and methods related to Examples I-V.

Mice: C57BL/6 (027, B6, H-2K^{h}, CD4+5.2), BALB/c (028, H-2K^{d}), and BDF1 (099, H-2K^{b/d}) were purchased from Charles River Laboratories. B6.129S7-Rag1^{tm1MomM}/J(002216, Rag1^{-/-}) mice, BDF1 (100006, B6D2F1/J), B6.Cg-Tg(Vil1cre)1000Gum/J mice(021504), and 129 (002448, 129S1/SvImJ) were purchased from the Jackson Laboratory. Taconic B6 (B6-F, C57BL/6NTac) and Taconic BALB/c (BALB-F, BALB/cAnNTac) were purchased from Taconic. Hif1a-floxed mice (Hif1a^{fl/fl}, C57BL/6 background)⁴⁰ containing the loxP site were crossed with Vil1cre mice (C57BL/6 background) to generate intestinal epithelial cells specific HIF1a-null mice (Hif1a^{fl/fl} Vil1-cre mice). Germ free (GF) C57BL/6 mice and Rag1−/− GF mice were raised and housed in ISOcage Positive isolators (Techniplast) at the germ-free mouse facility at the University of Michigan. Germ-free status was verified by aerobic, anaerobic cultures, and gram stain. 6-12 weeks old female mice used for experiments. All mice were kept under specific pathogen-free (SPF) conditions or GF conditions and cared for according to regulations reviewed and approved by the University of Michigan Committee on the Use and Care of Animals (PRO00009494), which are based on the University of Michigan Laboratory Animal Medicine guidelines. Mouse studies from MSKCC followed the respective Institutional Animal Care and Use Committee guidelines (99-07-025) and were kept under specific pathogen-free (SPF) conditions. B6 mice 6-8 weeks old were treated with 2 weeks of antibiotics cocktail (ampicillin 1 mg/ml (A9393, Sigma Aldrich)+kanamycin 1 mg/l (60615, Sigma Aldrich)+metronidazole 1 mg/ml (M1547, Sigma Aldrich)+vancomycin 0.5 mg/l (SBR00001, Sigma Aldrich) plus 3% stevia or ampicillin 1 mg/ml+neomycin 1 mg/ml+metronidazole 1 mg/ml+vancomycin 0.5 mg/ml in filtered double distillated drinking water. BMT recipients were orally treated with deferasirox (20 mg/kg, SML2673-50, Sigma Aldrich) and vehicle every day until day21 after BMT. For the co-housing experiments, mice were co-housed in a ratio of 1:1 naïve mice, B6 Ab, and BMT mice respectively. For FIG. 1A, Allo B6 and B6 were co-housed. For FIG. 1G, SynB6 or Allo B6 were co-housed with B6Ab. For FIG. 3A, the design of the experiment is same as FIG. 1A. For the fecal microbiota transplant, mice were gavaged by 10 doses of intestinal content from recipient mice for 2 weeks. Each gavage day one BMT mouse whole intestinal content was collected and homogenized in sterile PBS. 200 ul of the solution was gavaged to each recipient mouse.

Hematopoietic cell transplantation model: Transplantations were performed as previously described³¹. Briefly, Splenic T cells from donors were enriched, and T-cell-depleted BM (TCD-BM) was depleted of T cells by autoMACS (Miltenyi Biotec) utilizing CD90.2 microbeads (130-121-278, Miltenyi Biotec) or CD5 microbeads (130-049-301, Miltenyi Biotec). The details of HCT model were described in Table S1. The mice were randomly assigned to syngeneic, allogeneic or treatment groups in each experiment.

Colitis models: For the T-cell transfer induced colitis model, isolated splenic T cells from B6 mice were stained with DAPI (#422801, 1 μM, Biolenged), APC-Cy7 anti-CD4+(560246, GK1.5, 1:100, BD Biosciences, San Jose, CA), APC anti-CD25 (101910, 3C7, 1:100, Biolegend), FITC anti-CD4+4 (103006, IM7, 1:100, Biolegend) and PE anti-CD4+5RB (103308, C363-16A, 1:100, Biolegend). CD4++CD25-CD4+4-CD4+5RB^{hi} cells were sorted with the MoFlo Astrios cell sorter (Beckman Coulter) and intraperitoneally injected into Rag-1^{-/-} SPF or GF recipients.

Systemic and histopathological analysis of GVHD: Survival after HCT was monitored daily and assessed the degree of clinical GVHD weekly, as described in Table S2⁴¹. Histopathological analysis of the liver, gastrointestinal (GI) tract, and lung, which are the primary GVHD target organs, was performed as described utilizing a semi-quantitative scoring system implemented in a blinded manner by a single pathologist (C.L.)⁴². A pathology scoring of GVHD was used to assess the following abnormalities known to be associated with GVHD. Small intestine: villous blunting, crypt regeneration, loss of enterocyte brush border, luminal sloughing of cellular debri, crypt cell apoptosis, outright crypt destruction, and lamina propria lymphocytic infiltrate: colon: crypt regeneration, surface coloncytes, colonocyte vacuolization, surface colonocyte attenuation, crypt cell apoptosis, outright crypt destruction, and lamina propria lymphocytic infiltrate. The scoring system denoted 0 as normal, 0.5 as focal and rare, 1.0 as focal and mild, 2.0 as diffuse and mild, 3.0 as diffuse and moderate, and 4.0 as diffuse and severe. Scores were added to provide a total score for each specimen. Only after scoring was performed were codes broken and data compiled. After scoring, the codes were broken, and the data compiled.

DNA extraction, 16S rRNA gene sequencing and data analysis: The University of Michigan Microbiome Core extracted DNA and prepared and sequenced the amplicon libraries. DNA was extracted using an Eppendorf EpMotion liquid handling system and the Qiagen MagAttract PowerMicrobiome kit (previously MoBio PowerMag Microbiome, 27500-4-EP, Qiagen) kit and protocol. DNA (1 μl) was quantified with the Quant-iT PicoGreen dsDNA Assay kit (p7589, Invitrogen).

The University of Michigan Microbiome Core prepared and sequenced the amplicon libraries. Extracted DNA was amplified with dual-index primers targeting the V4 region of the 16S rRNA gene, as previously described 43 with the following PCR conditions: 2 min at 95° C., 30 cycles×[95° C. for 20 s, 55° C. for 15 s, and 72° C. for 5 min], followed by 72° C. for 10 min.

Libraries were prepared as previously described⁴⁴, with minor modifications: the final library concentration was 5.5 pM and 15% PhiX spike-in was added to increase diversity. Sequencing was performed on an Illumina MiSeq using the 500 cycles MiSeq Reagent Kit V2 (catalog no. MS-102-2003) with modifications described in the Schloss MiSeq SOP⁴³.

Paired-end 16S V4 sequences were processed using the software Mothur⁴⁵ (version 1.40.2 for first sequencing run, version 1.42.3 for subsequent run). The Schloss MiSeq SOP⁴³ as of August 2019 was followed to reduce PCR and sequencing errors. The sequences were aligned to a reference alignment based on SILVA release 132. After pre-clustering and chimera removal with vsearch, the remaining sequences (as well as the OTUs later) were classified to RDP taxonomy⁴⁶ based on RDP training set no 16⁴⁷. The bacterial 16S V4 sequences were phylotyped into genus bins to make community composition bar plots and clustered into 97% identity OTUs, with OTU abundance compiled for subsequent statistical analysis. These steps also followed the Schloss MiSeq SOP. Based on OTU abundance, PCoA was plotted and alpha diversity estimated with inverse Simpson. Differentially abundant taxa were determined with LEfSe version 1.1.2 48 cladograms and LDA scores of LEfSe results were plotted using the same software.

The relative abundance of bacterial OTUs grouped by oxygen sensitivity were compared by summing the relative abundance of known obligate anaerobes (Actinomyces, Bacteroides, Clostridium, Faecalibacterium, Blautia, Ruminococcus, Parabacteroides, and Bifidobacterium), facultative anaerobes (Escherichia/Shigella, Klebsiella, Salmonella, Enterococcus, Lactobacillus, and Staphylococcus), and unclassified. Differences in relative abundance between experimental groups were evaluated with Wilcoxon signed-rank tests using the ggpubr (version 0.4.0) and ggplot2⁴⁹ (version 3.3.5) R packages in R version 4.1.3. Unprocessed 16s rRNA sequencing reads were deposited at the NCBI Short Read Archive (SRA) and are accessible via BioProject PRJNA910578. The code used for analysis for 16S rRNA sequencing is available at https://doi.org/10.5281/zenodo.7401507.

Intestinal epithelial cells and intraepithelial cells isolation: Luminal contents from dissected colon and ileum were flushed with CMF buffer: Ca²⁺/Mg²⁺ free HBSS (14185052, Thermo Fisher Scientific) supplemented with 25 mM sodium bicarbonate (S6014, Sigma-Aldrich) and 2% FBS (100-106, Gemini Bio Products, USA). Intestines were then minced into 5 mm pieces, washed with CMF buffer four times, transferred to CMF with 5 mM EDTA (51201, Lonza), and incubated at 37° C. for 40 minutes (shaking tubes every 10 minutes). Supernatants containing IECs were then transferred through 100 μM cell filter followed by incubation on ice for 10 minutes to allow sedimentation. Supernatants were again transferred through a 75 μM cell filter.

Preparation of lymphocytes single cell suspension and flow cytometry: Systemic lymph nodes (axillary and inguinal), mesenteric lymph nodes and spleens were mechanically disrupted. Red blood cells were lysed (R7757, Sigma-Aldrich). For intra epithelial lymphocytes (IEL) isolation, intestines were minced in HBSS buffer (141850, Gibco) supplemented with 2.5% heat-inactivated FBS (100-106, Gibco) (HBSS+) and washed with magnetic stirring at 37° C. Intestine pieces were then incubated in HBSS+/1 mM DTT (DTT, Gold Biotechnology) at 37° C. followed by additional washes and incubation in HBSS+/1 mM EDTA (51201, Lonza). The supernatant was then layered on a 75%/40% Percoll Plus (17-5445, GE Healthcare) gradient to collect enriched IELs.

Single cell suspensions were resuspended in FACS wash buffer (2% bovine serum albumin in PBS). Cells were stained with conjugated monoclonal antibodies (mAbs): fluorescein isothiocyanate (FITC)-conjugated mAbs to CD8+(100705, clone 53-6.7, BioLegend), IL-17A (506907, clone TC11-18H10.1, BioLegend), CD25 (101907, clone 3c7, BioLegend), CD11c (117305, clone N418, BioLegend) and CD8+0 (104705, clone 16-10A1, Biolegend); phycoerythrin (PE)-conjugated mAbs to CD62L (104407, clone MEL-14, BioLegend), CD69 (104507, clone H1.2F3, BioLegend), IFNγ (505807, clone XMG1.2, BioLegend), FoxP3 (126403, clone MF-14, BioLegend), CD4+0 (553791, clone 3/23, BD Pharmingen), CD274 (124307, PD-L1, clone 10F9G2, BioLegend), IL-6 (504503, clone MP5-20F3, BioLegend); PerCP-Cy5.5 to CD4+5.2 (109827, clone 104, BioLegend): APC-conjugated mAbs to mouse CD4+4 (103011, clone IM7, BioLegend), CD279 (PD-1, 135209, clone 29F1.A12, BioLegend), IL4 (504105, clone 11B11, BioLegend), RORγt (17-6988-82, clone AFKJS-9, Invitrogen), CD11c (117309, clone N418, BioLegend), TNFα (506307, clone MP6-XT22, BioLegend); and APC-Cy7 to CD4+(100413, clone GK1.5, BioLegend). For cytokine analysis, cells were treated with RPMI containing 10% FBS, phorbol 12-myr-istate 13-acetate (PMA) (10 ng/mL, P1585, Sigma-Aldrich)/Ionomycin (1 mM, 13909, Sigma-Aldrich) cocktail or lipopolysaccharide (LPS, 500 ng/mL, L2654, Sigma-Aldrich) in the presence of protein transport inhibitor cocktail (X500, 00-4980-03, eBioscience) at 37° C. for 6 hours. For intracellular staining, cells were fixed with FoxP3 staining buffer set (FoxP3 and RORγt) or IC fixation buffer (for cytokines, 00-8222-49, eBioscience) and permeabilized with permeabilization buffer (00-8333-56, eBioscience) according to the manufacturer protocol. Cells were analyzed using the Attune NxT flow cytometer.

Immunoblot analysis: Isolated mitochondria or IECs were lysed in RIPA buffer (89901, Thermo Scientific). Equal amounts of proteins were loaded on 4-12% SDS-PAGE gel (NP0321, Invitrogen), electrophoresed and subsequently transferred to a PVDF membrane (ISEQ85R, Millipore) using a Bio-Rad semi-dry transfer cell (20 V, 1 h). Blots were incubated with anti-HIF1a (GTX127309, polyclonal, 1:1000, GeneTex), anti-PHD3 (NB100-139, 1:1000, Novus Biologicals), and anti-actin (8226, mAbcam8226, 1:3000, Abcam) primary antibodies overnight at 4° C. Incubation with secondary anti-rabbit-HRP (7074S, Cell Signaling Technology) was performed at room temperature for 1 hour. Bound antibody was detected using Super Signal ECL substrate (32106, Thermo Scientific) and quantitated using ChemiDoc MP Imaging system (BioRad). Densitometric analysis was performed using Image J⁵⁰ (v 1.53c).

Immunohistocheinistry staining: For Hif1α staining, colonic tissues were processed, embedded in paraffin, and cut into 5 μm sections. Slides were de-paraffinized, and heat-induced antigen retrieval was performed with 10 mM sodium citrate buffer. Endogenous peroxidases were quenched with 3.0% hydrogen peroxide for 15 min. Primary anti-Hif1α (NB100-479, Novus Biologicals) was diluted 1:200 in PBST containing 10% goat serum (16210064, Thermo Fisher Scientific) and incubated for 60 min at room temperature. Bound anti-body was detected using an anti-rabbit HRP labeled polymer (ab214880, abcam) incubated for 30 min and ImmPACT DAB (SK-4105, VECTOR labolatories). Slides were then counterstained with hematoxylin, dehydrated, and covered. For hypoxia staining, recipient mice were administered pimonidazole (PMDZ, HP7) from Hypoxyprobe, Inc. by intraperitoneal injection 30 min prior to sacrifice. Colon and ileum samples were paraffin-embedded and stained according to the manufacturer's instructions and counterstained with DAPI (P36931, Thermo Scientific)⁵¹.

Seahorse analysis: IECs were resuspended with complete seahorse XF assay medium (103335-100, Aglient) with 17.5 mM glucose (G7021, Sigma-Aldrich), 1 mM sodium pyruvate (S8636, Sigma-Aldrich), 2 mM glutamine (GLLO2, Caisson Labs), 2% BSA (BP1600-100, Fisher Scientific), 10 uM Y-27632 and 1% penicillin-streptomycin (516106, Sigma-Aldrich) adjusted to pH 7.4. Cells were plated at 8×10⁴ cells per well in a Seahorse assay plate, pretreated with matrigel (354262, Corning). Cells were equilibrated to 37° C. for 30 min before assay. Respiration profile was assessed in 96XF instrument with Mitostress assay as indicated upon cell treatment with 5 μM FCCP (ab120081, abcam), 7.5 μM oligomycin A (75351, Sigma-Aldrich), 4 M Rotenone (3616, Tocris), and 4 μM antimycin A (A8674, Sigma-Aldrich). Seahorse Wave Desktop Software (version 2.6.1.53) was used for data analysis.

Measurements of oxygen concentration in colonic mucosa: C57BL/6 mice post HCT day7 and day 21 were subject to be determined oxygen concentration levels in the colonic mucosa. The oxygen Pst1 optode microsensors (Presens) at the tip of a fiber optic cable were used. The optode was inserted into the mouse's colon at a depth of one to three cm via an endoscope (Karl Storz). Before oxygen measurements, mice were anesthetized with 4% isoflurane and then received 2% isoflurane during the oxygen measurement. The colon was insufflated with nitrogen gas to expunge both extraneous oxygen and oxygen that had leached into the rectum when insertion of the endoscope. Visual assessments of when the microsensor contacted with the colonic mucosa using a camera attached to the endoscope. Oxygen concentrations were recorded with a PreSens Microx TX3 Trace Micro fiber optic 02 transmitter and Presens' Oxyview TX3 v. 5.31 software using default parameters. Prior to measurements, a two-point calibration of the microsensor was conducted with air-saturated water and 100 ml of anoxic water that was attained by the addition of one gram of sodium sulfite (Acros Organics) and 50 L of 500 mM cobalt nitrate solution (Acros Organics). Mouse body was kept on a 37° C. heating pad with a Gaymar T-pump system. To prepare the colon for imaging, the colon was flushed with water. Oxygen concentration readings from the mucosa that remained stable for at least 30 seconds were recorded. Readings from three to five locations in the colon were then obtained for each mouse; readings from each location were subsequently averaged to obtain a mean oxygen concentration for each mouse. 0.2 RNA isolation and RT-PCR: RNA isolation and RT-PCR: Total RNA from single-cell suspensions was isolated using the RNeasy Kit (74104, QIAGEN) and reverse transcribed into cDNA using the High Capacity cDNA Reverse Transcription Kit (4374966, Applied Biosystems).

The following primers and PowerUP SYBR green polymerase were used to detect the following transcripts: 5′-TGACCTCAACTACATGGTCTACA-3′ (SEQ ID NO.: 1) and 5′-CTTCCCATTCTCGGCCTTG-3′ (SEQ ID NO.: 2) (Gapdh), 5′-CAGTCACCTGGTTGCTGCAA-3′ (SEQ ID NO.: 3) and 5′-CAGTCACCTGGTTGCTGCAA-3′ (SEQ ID NO.: 4) (Hif1α), 5′-TGCTGAAGAAAGGGCAGAAG-3′ (SEQ ID NO.: 5) and 5′-GCACACCACAGTCAGTCTTTA-3′ (SEQ ID NO.: 6) (Egln: Phd3). All reactions were performed according to manufacturer's instructions. All primers were verified for the production of a single specific PCR product via melting curve analysis.

Quantification of iron in intestinal content: The intestinal content in colon and ileum from allogeneic recipient mice were collected after euthanized. Then, the intestinal contents were processed according to the manufacturer's instruction of Iron Assay Kit (MAK025, Sigma-Aldrich).

Quantification and Statistical Analysis: All statistical analysis was performed using Graph Pad Prism (v8.0.0, Graph Pad Software Inc) and Excel2016 (version2105) to do the graph figures and statistics. P values <0.05 were considered as significant: P values >0.05 were considered as non-significant (* p<0.05, ** p<0.01, *** p<0.001 and **** p<0.0001). All sample sizes and statistical tests used are detailed in each figure legend. All replicates are biological replicates. No data were excluded. All experiments in vitro and in vivo were performed twice or more than twice. Data shown as ±SEM according to figure legend.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes.

The following references numerically denoted throughout the application are incorporated by references in their entireties:
1. Litvak, Y., Byndloss, M. X., and Baumler, A. J. (2018). Colonocyte metabolism shapes the gut microbiota. Science 362. 10.1126/science.aat9076.
2. Byndloss, M. X., Pernitzsch, S. R., and Baumler, A. J. (2018). Healthy hosts rule within: ecological forces shaping the gut microbiota. Mucosal Immunol 11, 1299-1305. 10.1038/s41385-018-0010-y.
3. Shono, Y., Docampo, M. D., Peled, J. U., Perobelli, S. M., Velardi, E., Tsai, J. J., Slingerland, A. E., Smith, O. M., Young, L. F., Gupta, J., et al. (2016). Increased GVHD-related mortality with broad-spectrum antibiotic use after allogeneic hematopoietic stem cell transplantation in human patients and mice. Sci Transl Med 8, 339ra371. 10.1126/scitranslmed.aaf2311.
4. Peled, J. U., Gomes, A. L. C., Devlin, S. M., Littmann, E. R., Taur, Y., Sung, A. D., Weber, D., Hashimoto, D., Slingerland, A. E., Slingerland, J. B., et al. (2020). Microbiota as Predictor of Mortality in Allogeneic Hematopoietic-Cell Transplantation. N Engl J Med 382, 822-834. 10.1056/NEJMoa1900623.
5. Caruso, R., Lo, B. C., and Nunez, G. (2020). Host-microbiota interactions in inflammatory bowel disease. Nat Rev Immunol 20, 411-426. 10.1038/s41577-019-0268-7.
6. Halfvarson, J., Brislawn, C. J., Lamendella, R., Vazquez-Baeza, Y., Walters, W. A., Bramer, L. M., D'Amato, M., Bonfiglio, F., McDonald, D., Gonzalez, A., et al. (2017). Dynamics of the human gut microbiome in inflammatory bowel disease. Nat Microbiol 2, 17004. 10.1038/nmicrobiol.2017.4.
7. Zeiser, R., and Blazar, B. R. (2017). Acute Graft-versus-Host Disease—Biologic Process, Prevention, and Therapy. N Engl J Med 377, 2167-2179. 10.1056/NEJMra1609337.
8. Bowerman, K. L., Varelias, A., Lachner, N., Kuns, R. D., Hill, G. R., and Hugenholtz, P. (2020). Continuous pre- and post-transplant exposure to a disease-associated gut microbiome promotes hyper-acute graft-versus-host disease in wild-type mice. Gut Microbes 11, 754-770. 10.1080/19490976.2019.1705729.
9. Shono, Y., and van den Brink, M. R. M. (2018). Gut microbiota injury in allogeneic haematopoietic stem cell transplantation. Nat Rev Cancer 18, 283-295. 10.1038/nrc.2018.10.
10. van Bekkum, D. W., Roodenburg, J., Heidt, P. J., and van der Waaij, D. (1974). Mitigation of secondary disease of allogeneic mouse radiation chimeras by modification of the intestinal microflora. J Natl Cancer Inst 52, 401-404. 10.1093/jnci/52.2.401.
11. Espey, M. G. (2013). Role of oxygen gradients in shaping redox relationships between the human intestine and its microbiota. Free Radic Biol Med 55, 130-140. 10.1016/j.freeradbiomed.2012.10.554.
12. Albenberg, L., Esipova, T. V., Judge, C. P., Bittinger, K., Chen, J., Laughlin, A., Grunberg, S., Baldassano, R. N., Lewis, J. D., Li, H., et al. (2014). Correlation between intraluminal oxygen gradient and radial partitioning of intestinal microbiota. Gastroenterology 147, 1055-1063 e1058. 10.1053/j.gastro.2014.07.020.
13. Donohoe, D. R., Garge, N., Zhang, X., Sun, W., O'Connell, T. M., Bunger, M. K., and Bultman, S. J. (2011). The microbiome and butyrate regulate energy metabolism and autophagy in the mammalian colon. Cell Metab 13, 517-526. 10.1016/j.cmet.2011.02.018.
14. Blouin, J. M., Penot, G., Collinet, M., Nacfer, M., Forest, C., Laurent-Puig, P., Coumoul, X., Barouki, R., Benelli, C., and Bortoli, S. (2011). Butyrate elicits a metabolic switch in human colon cancer cells by targeting the pyruvate dehydrogenase complex. Int J Cancer 128, 2591-2601. 10.1002/ijc.25599.
15. Colgan, S. P., and Taylor, C. T. (2010). Hypoxia: an alarm signal during intestinal inflammation. Nat Rev Gastroenterol Hepatol 7, 281-287. 10.1038/nrgastro.2010.39.
16. Mathewson, N. D., Jenq, R., Mathew, A. V., Koenigsknecht, M., Hanash, A., Toubai, T., Oravecz-Wilson, K., Wu, S. R., Sun, Y., Rossi, C., et al. (2016). Gut microbiome-derived metabolites modulate intestinal epithelial cell damage and mitigate graft-versus-host disease. Nat Immunol 17, 505-513. 10.1038/ni.3400.
17. Fujiwara, H., Docampo, M. D., Riwes, M., Peltier, D., Toubai, T., Henig, I., Wu, S. J., Kim, S., Taylor, A., Brabbs, S., et al. (2018). Microbial metabolite sensor GPR43 controls severity of experimental GVHD. Nat Commun 9, 3674. 10.1038/s41467-018-06048-w.
18. Kelly, C. J., Zheng, L., Campbell, E. L., Saeedi, B., Scholz, C. C., Bayless, A. J., Wilson, K. E., Glover, L. E., Kominsky, D. J., Magnuson, A., et al. (2015). Crosstalk between Microbiota-Derived Short-Chain Fatty Acids and Intestinal Epithelial HIF Augments Tissue Barrier Function. Cell Host Microbe 17, 662-671. 10.1016/j.chom.2015.03.005.
19. Byndloss, M. X., and Baumler, A. J. (2018). The germ-organ theory of non-communicable diseases. Nat Rev Microbiol 16, 103-110. 10.1038/nrmicro.2017.158.
20. Litvak, Y., Byndloss, M. X., Tsolis, R. M., and Baumler, A. J. (2017). Dysbiotic Proteobacteria expansion: a microbial signature of epithelial dysfunction. Curr Opin Microbiol 39, 1-6. 10.1016/j.mib.2017.07.003.
21. Fujiwara, H., Seike, K., Brooks, M. D., Mathew, A. V., Kovalenko, I., Pal, A., Lee, H. J., Peltier, D., Kim, S., Liu, C., et al. (2021). Mitochondrial complex II in intestinal epithelial cells regulates T cell-mediated immunopathology. Nat Immunol 22, 1440-1451. 10.1038/s4t590-021-01048-3.
22. Stein-Thoeringer, C. K., Nichols, K. B., Lazrak, A., Docampo, M. D., Slingerland, A. E., Slingerland, J. B., Clurman, A. G., Armijo, G., Gomes, A. L. C., Shono, Y., et al. (2019). Lactose drives Enterococcus expansion to promote graft-versus-host disease. Science 366, 1143-1149. 10.1126/science.aax3760.
23. Jenq, R. R., Ubeda, C., Taur, Y., Menezes, C. C., Khanin, R., Dudakov, J. A., Liu, C., West, M. L., Singer, N. V., Equinda, M. J., et al. (2012). Regulation of intestinal inflammation by microbiota following allogeneic bone marrow transplantation. J Exp Med 209, 903-911. 10.1084/jem.20112408.
24. Glover, L. E., Lee, J. S., and Colgan, S. P. (2016). Oxygen metabolism and barrier regulation in the intestinal mucosa. J Clin Invest 126, 3680-3688. 10.1172/JCI84429.
25. Ibrahim, A. S., Gebermariam, T., Fu, Y., Lin, L., Husseiny, M. I., French, S. W., Schwartz, J., Skory, C. D., Edwards, J. E., Jr., and Spellberg, B. J. (2007). The iron chelator deferasirox protects mice from mucormycosis through iron starvation. J Clin Invest 117, 2649-2657. 10.1172/JC132338.
26. Packey, C. D., Shanahan, M. T., Manick, S., Bower, M. A., Ellermann, M., Tonkonogy, S. L., Carroll, I. M., and Sartor, R. B. (2013). Molecular detection of bacterial contamination in gnotobiotic rodent units. Gut Microbes 4, 361-370. 10.4161/gmic.25824.
27. Toubai, T., Fujiwara, H., Rossi, C., Riwes, M., Tamaki, H., Zajac, C., Liu, C., Mathew, A. V., Byun, J., Oravecz-Wilson, K., et al. (2019). Host NLRP6 exacerbates graft-versus-host disease independent of gut microbial composition. Nat Microbiol 4, 800-812. 10.1038/s4t564-019-0373-1.
28. Song, X., Sun, X., Oh, S. F., Wu, M., Zhang, Y., Zheng, W., Geva-Zatorsky, N., Jupp, R., Mathis, D., Benoist, C., and Kasper, D. L. (2020). Microbial bile acid metabolites modulate gut RORgamma(+) regulatory T cell homeostasis. Nature 577, 410-415. 10.1038/s41586-019-1865-0.
29. Sato, Y., Atarashi, K., Plichta, D. R., Arai, Y., Sasajima, S., Kearney, S. M., Suda, W., Takeshita, K., Sasaki, T., Okamoto, S., et al. (2021). Novel bile acid biosynthetic pathways are enriched in the microbiome of centenarians. Nature 599, 458-464. 10.1038/s41586-021-03832-5.
30. Campbell, C., McKenney, P. T., Konstantinovsky, D., Isaeva, O. I., Schizas, M., Verter, J., Mai, C., Jin, W. B., Guo, C. J., Violante, S., et al. (2020). Bacterial metabolism of bile acids promotes generation of peripheral regulatory T cells. Nature 581, 475-479. 10.1038/s41586-020-2193-0.
31. Reddy, P., Sun, Y., Toubai, T., Duran-Struuck, R., Clouthier, S. G., Weisiger, E., Maeda, Y., Tawara, I., Krijanovski, O., Gatza, E., et al. (2008). Histone deacetylase inhibition modulates indoleamine 2,3-dioxygenase-dependent DC functions and regulates experimental graft-versus-host disease in mice. J Clin Invest 118, 2562-2573. 10.1172/JC134712.
32. Toubai, T., Hou, G., Mathewson, N., Liu, C., Wang, Y., Oravecz-Wilson, K., Cummings, E., Rossi, C., Evers, R., Sun, Y., et al. (2014). Siglec-G-CD24 axis controls the severity of graft-versus-host disease in mice. Blood 123, 3512-3523. 10.1182/blood-2013-12-545335.
33. Tamburini, F. B., Andermann, T. M., Tkachenko, E., Senchyna, F., Banaei, N., and Bhatt, A. S. (2018). Precision identification of diverse bloodstream pathogens in the gut microbiome. Nat Med 24, 1809-1814. 10.1038/s41591-018-0202-8.
34. Semenza, G. L. (2012). Hypoxia-inducible factors in physiology and medicine. Cell 148, 399-408. 10.1016/j.cell.2012.01.021.
35. Karhausen, J., Furuta, G. T., Tomaszewski, J. E., Johnson, R. S., Colgan, S. P., and Haase, V. H. (2004). Epithelial hypoxia-inducible factor-1 is protective in murine experimental colitis. J Clin Invest 114, 1098-1106. 10.1172/JC121086.
36. Shah, Y. M., Ito, S., Morimura, K., Chen, C., Yim, S. H., Haase, V. H., and Gonzalez, F. J. (2008). Hypoxia-inducible factor augments experimental colitis through an MIF-dependent inflammatory signaling cascade. Gastroenterology 134, 2036-2048, 2048 e2031-2033. 10.1053/j.gastro.2008.03.009.
37. Xu, M., Tao, J., Yang, Y., Tan, S., Liu, H., Jiang, J., Zheng, F., and Wu, B. (2020). Ferroptosis involves in intestinal epithelial cell death in ulcerative colitis. Cell Death Dis 11, 86. 10.1038/s41419-020-2299-1.
38. Xu, S., He, Y., Lin, L., Chen, P., Chen, M., and Zhang, S. (2021). The emerging role of ferroptosis in intestinal disease. Cell Death Dis 12, 289. 10.1038/s41419-021-03559-1.
39. Ellermann, M., and Arthur, J. C. (2017). Siderophore-mediated iron acquisition and modulation of host-bacterial interactions. Free Radic Biol Med 105, 68-78. 10.1016/j.freeradbiomed.2016.10.489.
40. Shah, Y. M., Matsubara, T., Ito, S., Yim, S. H., and Gonzalez, F. J. (2009). Intestinal hypoxia-inducible transcription factors are essential for iron absorption following iron deficiency. Cell Metab 9, 152-164. 10.1016/j.cmet.2008.12.012.
41. Cooke, K. R., Hill, G. R., Crawford, J. M., Bungard, D., Brinson, Y. S., Delmonte, J., Jr., and Ferrara, J. L. (1998). Tumor necrosis factor-alpha production to lipopolysaccharide stimulation by donor cells predicts the severity of experimental acute graft-versus-host disease. J Clin Invest 102, 1882-1891. 10.1172/JC14285.
42. Hill, G. R., Cooke, K. R., Teshima, T., Crawford, J. M., Keith, J. C., Jr., Brinson, Y. S., Bungard, D., and Ferrara, J. L. (1998). Interleukin-11 promotes T cell polarization and prevents acute graft-versus-host disease after allogeneic bone marrow transplantation. J Clin Invest 102, 115-123. 10.1172/JC13132.
43. Kozich, J. J., Westcott, S. L., Baxter, N. T., Highlander, S. K., and Schloss, P. D. (2013). Development of a dual-index sequencing strategy and curation pipeline for analyzing amplicon sequence data on the MiSeq Illumina sequencing platform. Appl Environ Microbiol 79, 5112-5120. 10.1128/AEM.01043-13.
44. Koenigsknecht, M. J., Theriot, C. M., Bergin, I. L., Schumacher, C. A., Schloss, P. D., and Young, V. B. (2015). Dynamics and establishment of Clostridium difficile infection in the murine gastrointestinal tract. Infect Immun 83, 934-941. 10.128/IAI.02768-14.
45. Schloss, P. D., Westcott, S. L., Ryabin, T., Hall, J. R., Hartmann, M., Hollister, E. B., Lesniewski, R. A., Oakley, B. B., Parks, D. H., Robinson, C. J., et al. (2009). Introducing mothur: open-source, platform-independent, community-supported software for describing and comparing microbial communities. Appl Environ Microbiol 75, 7537-7541. 10.1128/AEM.01541-09.
46. Wang, Q., Garrity, G. M., Tiedje, J. M., and Cole, J. R. (2007). Naive Bayesian classifier for rapid assignment of rRNA sequences into the new bacterial taxonomy. Appl Environ Microbiol 73, 5261-5267. 10.1128/AEM.00062-07.
47. Cole, J. R., Wang, Q., Fish, J. A., Chai, B., McGarrell, D. M., Sun, Y., Brown, C. T., Porras-Alfaro, A., Kuske, C. R., and Tiedje, J. M. (2014). Ribosomal Database Project: data and tools for high throughput rRNA analysis. Nucleic Acids Res 42, D633-642. 10.1093/nar/gkt1244.
48. Segata, N., Izard, J., Waldron, L., Gevers, D., Miropolsky, L., Garrett, W. S., and Huttenhower, C. (2011). Metagenomic biomarker discovery and explanation. Genome Biol 12, R60. 10.1186/gb-2011-12-6-r60.
49. Wickham, H. (2016). ggplot2: Elegant Graphics for Data Analysis. Use R!. 2nd ed. Springer International Publishing: Imprint: Springer.
50. Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682. 10.1038/nmeth.2019.
51. Campbell, E. L., Bruyninckx, W. J., Kelly, C. J., Glover, L. E., McNamee, E. N., Bowers, B. E., Bayless, A. J., Scully, M., Saeedi, B. J., Golden-Mason, L., et al. (2014). Transmigrating neutrophils shape the mucosal microenvironment through localized oxygen depletion to influence resolution of inflammation. Immunity 40, 66-77. 10.1016/j.immuni.2013.11.020.
52. Kim, Y. G., Sakamoto, K., Seo, S. U., Pickard, J. M., Gillilland, M. G., 3rd, Pudlo, N. A., Hoostal, M., Li, X., Wang, T. D., Feehley, T., et al. (2017). Neonatal acquisition of Clostridia species protects against colonization by bacterial pathogens. Science 356, 315-319. 10.1126/science.aag2029.

1. A method for treating, preventing and/or ameliorating symptoms related to one or more conditions associated with and/or related to impaired intestinal physiological hypoxia in a human subject comprising administering to the subject a therapeutically effective amount of a pharmaceutical composition comprising one or more agents capable of restoring intestinal physiological hypoxia.

2. (canceled)

3. The method of claim 1, wherein the condition associated with and/or related to impaired intestinal physiological hypoxia is one or more of: intestinal dysbiosis, an immune mediated intestinal disease, idiopathic inflammatory bowel disease (IBD), graft versus host disease (GVHD), enteropathy in primary immunodeficiency, allogeneic hematopoietic stem cell transplantation (HSCT), decreased obligate anaerobes in the subject's microbiome, T-cell mediated metabolic defect in intestinal epithelial cells, and a dysbiosis inducing event.

4-9. (canceled)

10. The method of claim 3, wherein the dysbiosis inducing event is one or more of:
treatment with one or more antibiotics (e.g., vancomycin) in connection with surgery;
an infectious disease;
a primary infection by Clostridium difficile;
a secondary or recurring infection by Clostridium difficile; and
traveler's diarrhea.

11. The method of claim 1, wherein the agent capable of restoring intestinal physiological hypoxia is an iron binding protein.

12. The method of claim 11, wherein the iron binding protein is selected from the group consisting of an iron chelator, lactoferrin, transferrin, ferritin, Ferric uptake repressor (FUR) protein, calcineurin, acid phosphatase and ferredoxin.

13. (canceled)

14. The method of claim 12, wherein the iron chelator is selected from the group consisting of desferrioxamine, Deferasirox, FBS0701, phenanthroline, ethylene diamine tetra-acetic acid (EDTA), diethylene triamine-pentaacetic acid (DTPA) and N,N′-bis[2-hydroxybenzoyl]ethylene diamine-N,N′-diacetic acid (HBED).

15. The method of claim 1, wherein the agent capable of restoring intestinal physiological hypoxia is any pharmaceutical agent capable of restoring intestinal eubiosis in the subject.

16. The method of claim 1, wherein the pharmaceutical agent is administered orally and/or by oral gavage.

17. The method of claim 1, wherein the method further comprises administering to the subject one or more of the following additional therapeutic agents: disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors.

18. The method of claim 17, wherein the additional therapeutic agents include one or more of: infliximab, adalimumab, etanercept, parenteral gold, and oral gold.

19-75. (canceled)

76. A kit comprising one or more agents capable of restoring intestinal physiological hypoxia and one or more additional therapeutic agents.

77. The kit of claim 76, wherein the agent capable of restoring intestinal physiological hypoxia is an iron binding protein, wherein the iron binding protein is selected from the group consisting of lactoferrin, transferrin, ferritin, Ferric uptake repressor (FUR) protein, calcineurin, acid phosphatase and ferredoxin.

78. (canceled)

79. The kit of claim 77, wherein the iron binding protein is an iron chelator, wherein the iron chelator is selected from the group consisting of desferrioxamine, Deferasirox, FBS0701, phenanthroline, ethylene diamine tetra-acetic acid (EDTA), diethylene triamine-pentaacetic acid (DTPA) and N,N′-bis[2-hydroxybenzoyl]ethylene diamine-N,N′-diacetic acid (HBED).

80. (canceled)

81. The kit of claim 76, wherein the agent capable of restoring intestinal physiological hypoxia is any pharmaceutical agent capable of restoring intestinal eubiosis in the subject.

82. The kit of claim 76, wherein the one or more additional therapeutic agents are selected from disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors.

83. (canceled)


SEQUENCE LISTING (6 sequences)

SEQ ID NO: 1 - DNA, 23 bp, Synthetic construct
  source 1..23: mol_type = other DNA
tgacctcaactacatggtctaca

SEQ ID NO: 2 - DNA, 19 bp, Synthetic construct
  source 1..19: mol_type = other DNA
cttcccattctcggccttg

SEQ ID NO: 3 - DNA, 20 bp, Synthetic construct
  source 1..20: mol_type = other DNA
cagtcacctggttgctgcaa

SEQ ID NO: 4 - DNA, 20 bp, Synthetic construct
  source 1..20: mol_type = other DNA
cagtcacctggttgctgcaa

SEQ ID NO: 5 - DNA, 20 bp, Synthetic construct
  source 1..20: mol_type = other DNA
tgctgaagaaagggcagaag

SEQ ID NO: 6 - DNA, 21 bp, Synthetic construct
  source 1..21: mol_type = other DNA
gcacaccacagtcagtcttta